(19) United States
(12) Patent Application Publication
(10) Pub. No.: US 2024/0239823 A1
(43) Pub. Date: Jul. 18, 2024

(54) PHOSPHINATE ESTER-CONTAINING DYES HAVING TUNABLE PROPERTIES AND METHODS OF MAKING THE SAME

(71) Applicants: University of Virginia Patent Foundation, Charlottesville, VA (US); NUTECH VENTURES, Lincoln, NE (US)

(72) Inventors: Cliff STAINS, Crozet, VA (US); Yuan FANG, Charlottesville, VA (US); Ruwen YIN, Charlottseville, VA (US); Frederik BRONDSTED, Charlottesville, VA (US); Xinqi ZHOU, Charlottesville, VA (US)

(21) Appl. No.: 18/556,703

(22) PCT Filed: May 10, 2022
(86) PCT No.: PCT/US2022/072230

§ 371 Date: Oct. 23, 2023

Related U.S. Application Data
(60) Provisional application No. 63/186,414, filed on May 10, 2021.

(51) Int. Cl. C07F 9/6584; A61K 47/54; A61K 49/00; C09B 11/28

(52) U.S. Cl. CPC C07F 9/65846; A61K 47/548; A61K 49/0021; C09B 11/28

(57) ABSTRACT

In one aspect, the disclosure relates to xanthene-, thiazine-, and oxazine-based dyes containing a phosphinate ester group and having near-infrared (NIR) absorption and methods of making the same. The fluorescence lifetimes and stabilities of the dyes can be tuned by modifying the molecule cores, making them suitable for a variety of chemical labeling, imaging, and other theranostic applications. This abstract is intended as a scanning tool for purposes of searching in the particular art and is not intended to be limiting of the present disclosure.

### CROSS REFERENCE TO RELATED APPLICATIONS

This application claims the benefit of U.S. Provisional Application No. 63/186,414 filed on May 10, 2021, which is incorporated herein by reference in its entirety.

### STATEMENT REGARDING FEDERALLY SPONSORED RESEARCH OR DEVELOPMENT

This invention was made with government support under grant number GM119751, awarded by the National Institutes of Health. The government has certain rights in the invention.

### BACKGROUND

Near-Infrared (NIR) dyes that absorb above 650 nm have attracted significant attention in the imaging community. The NIR absorbance of this type of dyes enables advanced, deep-tissue imaging in organisms, including humans. However, many NIR dyes suffer from lack of chemical stability in water or biological fluids or are otherwise unsuitable for many imaging applications due to short emission lifetimes or lack of sufficient brightness to achieve good image depth and resolution.

It would be desirable if NIR dyes could be developed having tunable stability for imaging and/or therapeutic cargo-delivery applications. The dyes would ideally be bright and have long, stable emissions lifetimes as well as having stability tunable with respect to the biochemistry of target cells. Current methods of 3D medical imaging, including X-ray and positron emission tomography (PET) require subjecting patients to ionizing radiation, which can be harmful to patient health over time, especially where multiple scans are required to monitor the progress of a treatment or condition. It would further be desirable if the dyes could produce a photoacoustic signal for use as photoacoustic probes in 3D imaging applications without the need to use ionizing radiation. These needs and other needs are satisfied by the present disclosure.

### SUMMARY

In accordance with the purpose(s) of the present disclosure, as embodied and broadly described herein, the disclosure, in one aspect, relates to xanthene-, oxazine-, and thiazine-based dyes containing a phosphinate ester group and having near-infrared (NIR) absorption and methods of making the same. The fluorescence lifetimes and stabilities of the dyes can be tuned by modifying the molecule cores, making them suitable for a variety of chemical labeling, imaging, and other theranostic applications.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1.1 shows a series of xanthene-based phosphinate ester-containing dyes that display NIR absorption. Photophysical properties are shown along with phosphinate ester half-lives in water at pH=7.4 (t_{1/2}). Phosphinate ester half-life can be tuned from ˜30 min (see NR₇₀₀) to 8.5 hrs (see NR₆₉₇) by leveraging structural modifications to the dye core. This has implications for stabilization of cargo for theranostic development as well as development of imaging agents.

FIG. 1.2 shows a series of xanthene-based phosphinate-containing dyes that display NIR absorption. Photophysical properties are shown. Modifications to the dye core result in brighter phosphinate dyes. Compare NR₆₆₇, NR₆₇₇, and NR₆₇₂ to NR₆₆₆.

FIG. 1.3 shows bulky ether groups decrease the rate of phosphinate ester hydrolysis. The bulkier NR₆₉₇ phosphinate ester leads to increased stability in water compared to NR₇₀₀ as assessed by stability of fluorescence emission. Thus, stable phosphinate ester dyes can be obtained by increasing the bulk of the ether substituent. Stability of the fluorescence emission is measured over time. This has implications for stabilization of cargo for theranostic development as well as development of imaging agents.

FIG. 1.4 shows one possible strategy for developing theranostics based on phosphinate-containing dyes. A non-fluorescent version of an NR dye containing a selective analyte recognition element (X) and a payload attached to the phosphinate ester is shown on the left. Reaction with a target analyte induces hydrolysis of the phosphinate ester and concomitant release of the payload and formation of a fluorescent NR dye (right).

FIG. 1.5 shows phosphinate-containing dyes are capable of producing photoacoustic signal. Photoacoustic signal (OA Signal) generation from the indicated NR dyes is plotted against their respective normalized absorption spectra. Clear evidence of photoacoustic signal generation is seen. Measurements were recorded in tissue phantoms at a depth of 1.5 cm on a MSOT inVision 128 instrument.

FIG. 2.1 shows a thiazine-based phosphinate-containing dye that displays NIR absorption. The structure of NR₇₅₁, a phosphinate-containing thiazine-based is shown with its optical properties.

FIGS. 2.2A-2.2E show that NR₇₅₁ displays significantly reduced aggregation in aqueous solutions. FIG. 2.2A: The structure of methylene blue (MB) and NR₇₅₁. Absorbance versus concentration for MB (FIG. 2.2B) and NR₇₅₁ (FIG. 2.2C) in water demonstrates a clear deviation from linearity for MB but not NR₇₅₁. FIG. 2.2D: Normalized absorbance spectra of increasing concentrations of MB and NR₇₅₁ in water demonstrate H-aggregate formation in MB as evidenced by an increase in the blue-shifted absorbance band. FIG. 2.2E: Addition of 0.1% SDS to MB reduces the intensity of the blue-shifted H-Aggregate absorption band in water. No change is observed in the absorbance of NR₇₅₁ under identical conditions.

FIGS. 2.3A-2.3B show that NR₇₅₁ displays solvent-dependent absorption and emission properties. Normalized absorption (FIG. 2.3A, top) and emission (FIG. 2.3A, bottom) spectra of NR₇₅₁ in the indicated solvents. A plot of the relationship between absorption as well as emission maxima and relative solvent polarity is shown (FIG. 2.3B).

FIG. 2.4 shows Az-NR₇₅₁ displays enhanced fluorescence brightness. The structure of Az-NR₇₅₁ (left) along with normalized absorption and emission spectra (right). Photophysical properties of Az-NR₇₅₁ and NR₇₅₁ are shown (bottom table), demonstrating an increase in fluorescence brightness of Az-NR₇₅₁.

FIG. 3.1 shows structures of NR dyes bearing bulky substituents on the pendant phenyl ring and their respective photophysical properties and hydrolysis half-lives in water at pH=7.4 (t_{1/2}).

FIG. 3.2 shows NR₇₀₀ displays a decrease in absorbance in the presence of glutathione (GSH).

FIG. 3.3 shows bulky substituents on the pendant phenyl ring protect NR dyes from quenching by GSH. The absorbance of NR₇₀₈ in the absence or presence of GSH is shown.

FIG. 4.1 shows structures of amine-bearing NR dyes and their respective photophysical properties and hydrolysis half-lives in water at pH=7.4 (t_{1/2}).

FIG. 5.1 shows a proof-of-principle for payload delivery using a phosphinate containing dye. Top: upon treatment with HOCl a dark compound is activated to form a fluorescent NR dye and release a fluorescent payload (coumarin). Bottom: spectroscopic measurements of the rate of NR dye formation (squares) and payload release (circles).

FIGS. 5.2A-5.2C show phosphinate-containing dyes as gated small molecule delivery vehicles for cytotoxic compounds in cells. FIG. 5.2A: The structure of NR—HOCl-1541B (left). Selective reaction with HOCl results in the gated release of 1541B, a cytotoxic compound, and a fluorescent NR dye (right) FIG. 5.2B: Viability of RAW 264.7 macrophages in the presence or absence of stimulation to produce endogenous HOCl (LPS and PMA), cells were incubated with 50 μM 1541B or NR—HOCl-1541B where indicated. FIG. 5.2C: Confocal imaging of RAW 264.7 cells incubated with 50 μM NR—HOCl-1541B in the presence or absence of stimulation to produce endogenous HOCl. Clear evidence of fluorescence, and payload delivery, is observed in cells producing HOCl.

FIG. 6.1 shows SNR₇₀₀—HOCl and its reaction with HOCl to produce a stable, photoacoustic (PA) active and fluorescent dye.

FIG. 6.2 shows turn-on absorbance and fluorescence of SNR₇₀₀—HOCl in the presence of HOCl.

FIG. 6.3 shows turn-on fluorescence of SNR₇₀₀—HOCl is selective for HOCl (1 eq.) compared to off-target reactive oxygen species (ROS) or reactive nitrogen species (RNS) (10 eq.).

FIG. 6.4 shows turn-on PA signal from SNR₇₀₀—HOCl in the presence of increasing equivalents of HOCl in a tissue phantom. The inset shows PA images of tissue phantoms.

FIGS. 7.1A-7.1C show imaging of orthotopic prostate tumors in mice with NR₆₆₆. FIG. 7.1A: A cartoon of HSA-NR₆₆₆. NIR fluorescence (FIG. 7.1B) or bioluminescence (FIG. 7.1C) images of mice bearing orthotopic, luciferase expressing PC3 tumors at day 40 post implantation (areas of fluorescence and/or bioluminescence circled). Fluorescence images were obtained after injection with HSA-NR₆₆₆. Clear colocalization of fluorescence and bioluminescence is observed.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

In one aspect, it is disclosed herein that the phosphinate functional group can dramatically red-shift the absorbance of commonly used dyes from the visible region into the NIR region (phosphinate installation generally red-shifts absorption and emission by ˜110 nm). Herein, in one aspect, are provided a series of phosphinate-containing dyes displaying absorbance in the NIR. In a further aspect, these new dyes have potential applications in fluorescence imaging, photoacoustic imaging, and the development of theranostic reagents.

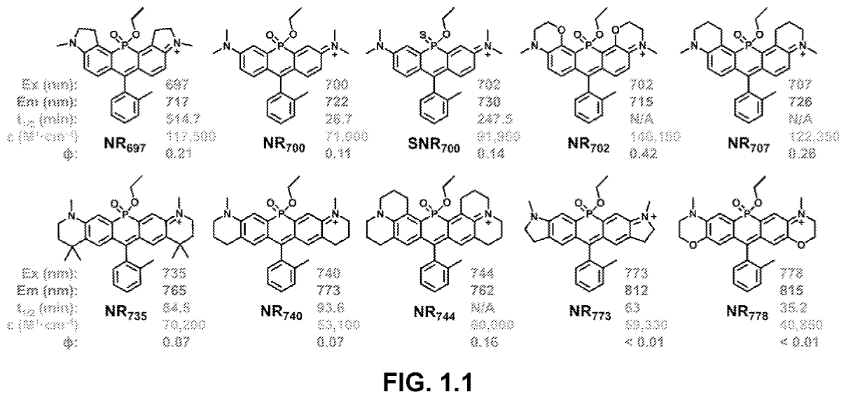

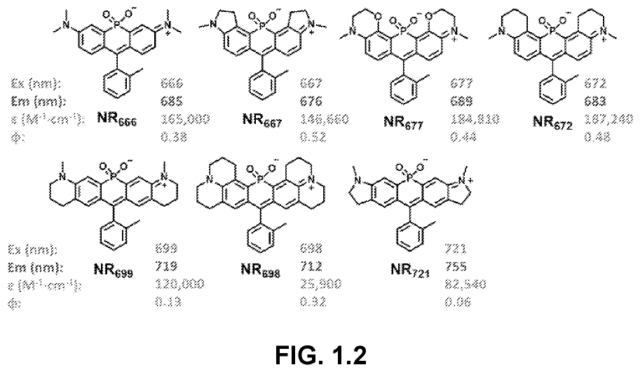

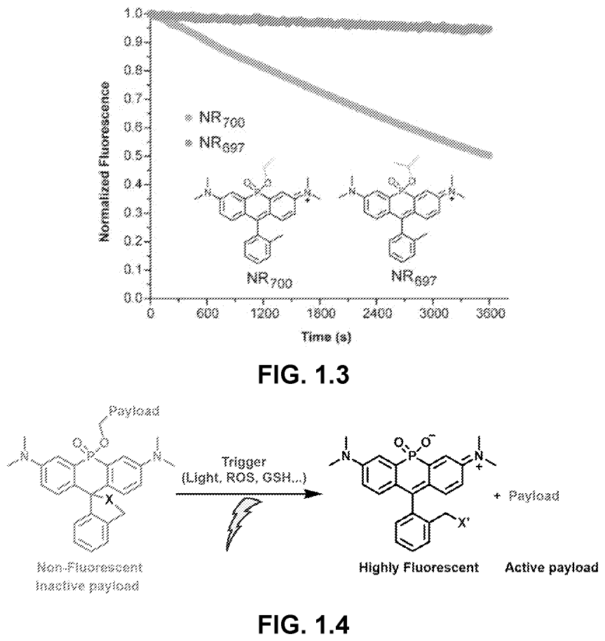

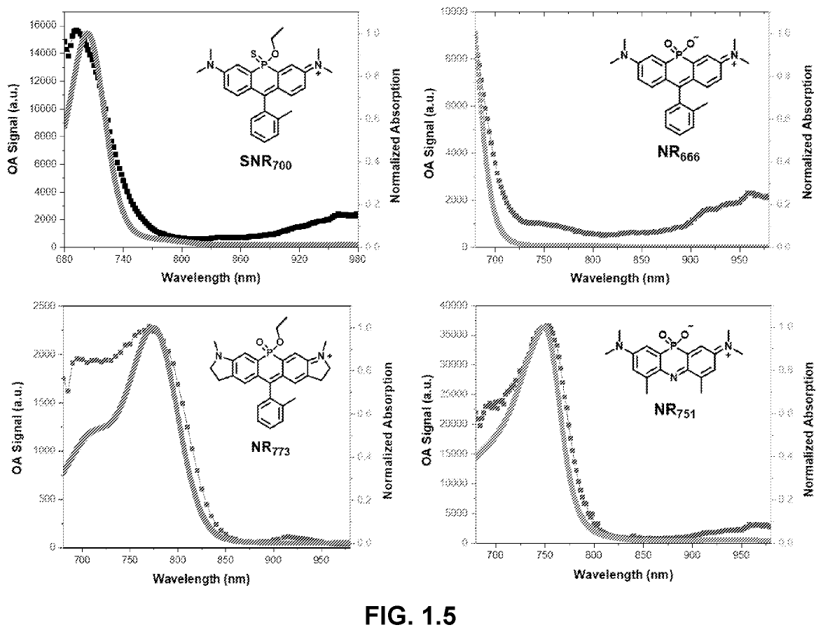

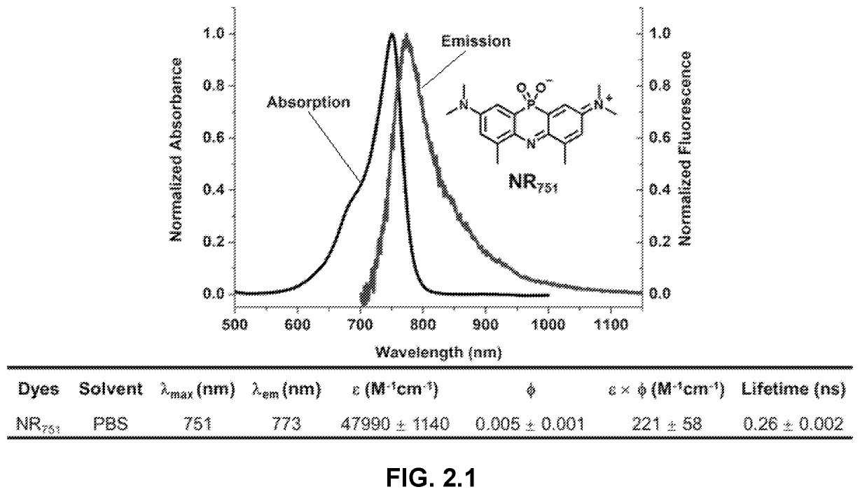

In one aspect, disclosed herein is a new series of NIR-absorbing xanthenes useful as further red-shifted dyes (herein, Nebraska Red or NR dyes) (FIGS. 1.1 and 1.2). In another aspect, molecules in the resulting series display absorbance (also called excitation) from ˜650-775 nm. In another aspect, these dyes have potential applications in deep-tissue imaging using fluorescence or photoacoustic readouts.

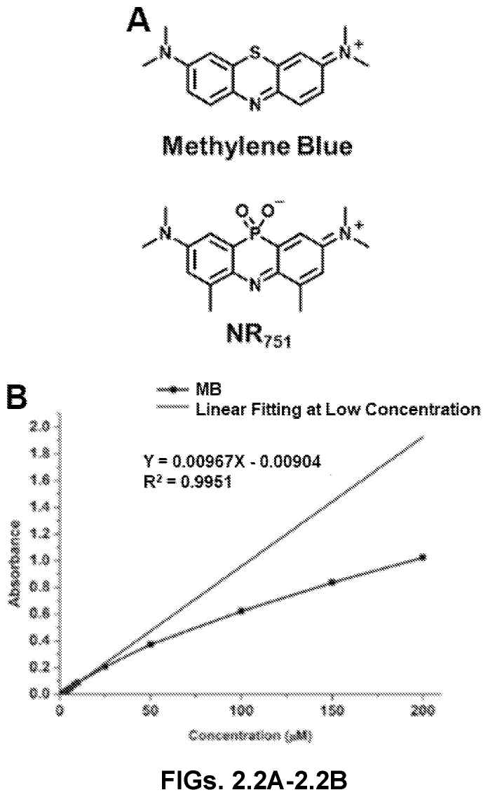

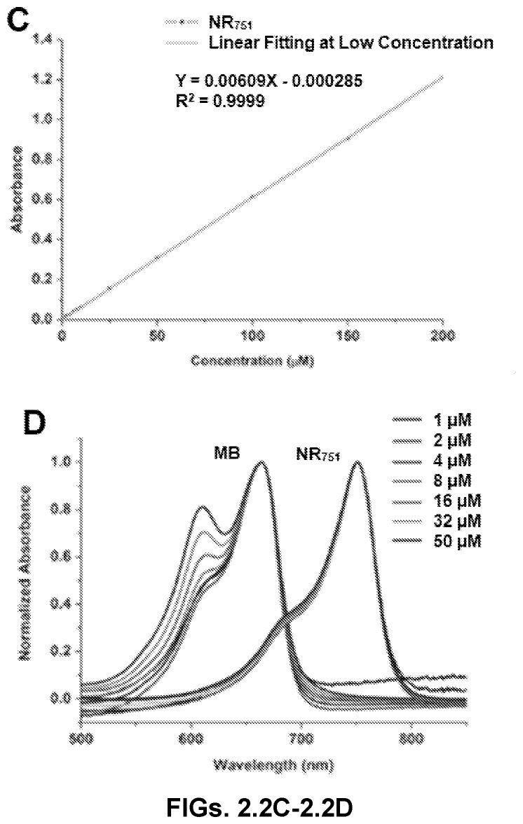

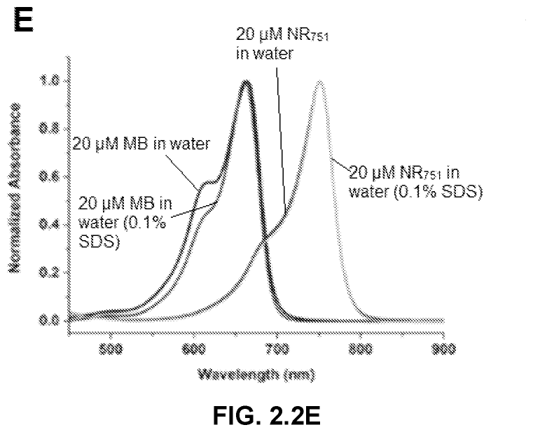

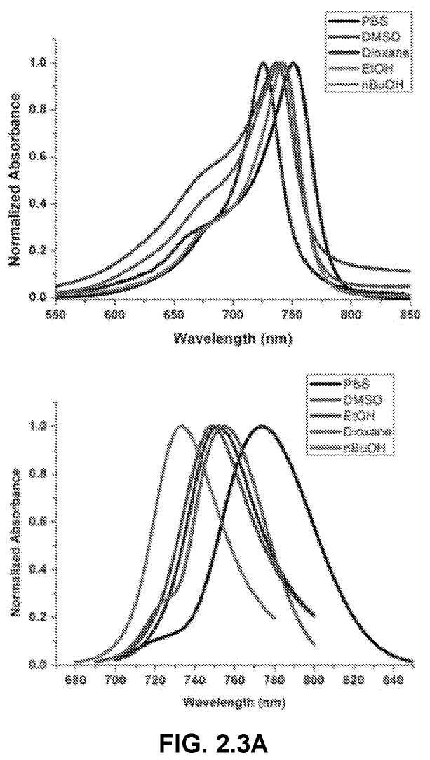

Also disclosed herein is a phosphinate-containing oxazine fluorophore (FIG. 2.1). In one aspect, this dye displays an ˜110 nm red-shift in absorption and emission compared to the parent oxazine (containing oxygen in place of the phosphinate) and demonstrates the generalizability of phosphinates for red-shifting the absorption and emission of dyes. In a further aspect, it is believed that structural modifications to this scaffold will produce dyes with absorption ranging from ˜800-900 nm.

In one aspect, disclosed herein is a dye having a structure of Formula I or an ionized form thereof:

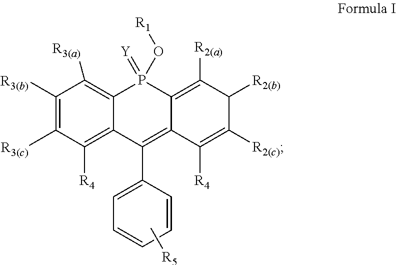

wherein R₁ is H, a C1-C6 linear or branched alkyl group, azide, alkyne, cyclooctyne, tetrazine, NHS ester, isocyanate, maleimide, iodoacetamide, diazonium salt, DNA-labeling agent, RNA-labeling agent, protein-labeling agent, or a 3-8-membered aryl, heteroaryl, cycloalkyl, or heterocycloalkyl group;
wherein R₂₍ₐ₎ is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{2(b)};
wherein R_{2(b)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, imine, or sulfonate, or independently forms a 5- or 6-membered aryl, cycloalkyl, or heterocycloalkyl group with R₂₍ₐ₎ or R_{2(c)}, or independently forms 5- or 6-membered aryl, cycloalkyl, or heterocycloalkyl groups with both R₂₍ₐ₎ and R_{2(c)};
wherein R_{2(c)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{2(b)};
wherein R₃₍ₐ₎ is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{3(b)};
wherein R_{3(b)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or independently forms a 5- or 6-membered aryl, cycloalkyl, or heterocycloalkyl group with R₃₍ₐ₎ or R_{3(c)}, or forms 5- or 6-membered aryl, cycloalkyl, or heterocycloalkyl groups with both R₃₍ₐ₎ and R_{3(c)};
wherein R_{3(c)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{3(b)};
wherein each R₄ is, independently, selected from hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate;
wherein each R₅ is, independently, selected from hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, C1-C4 alkoxy, amide, substituted amide, or sulfonate; and
wherein Y is O or S.

In another aspect, the structure of Formula I or ionized form thereof can be selected from:

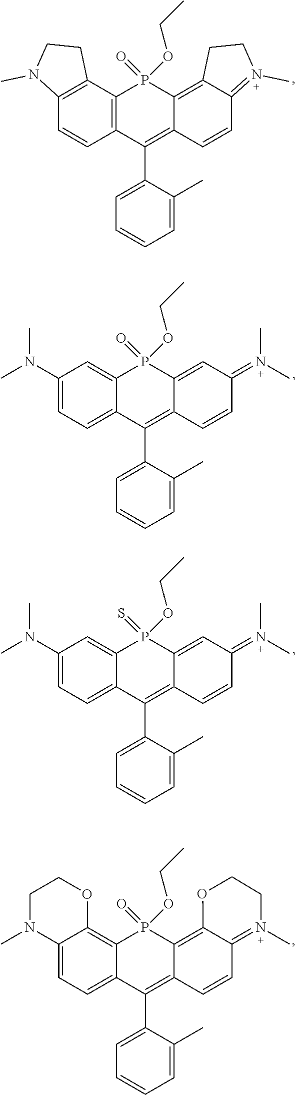

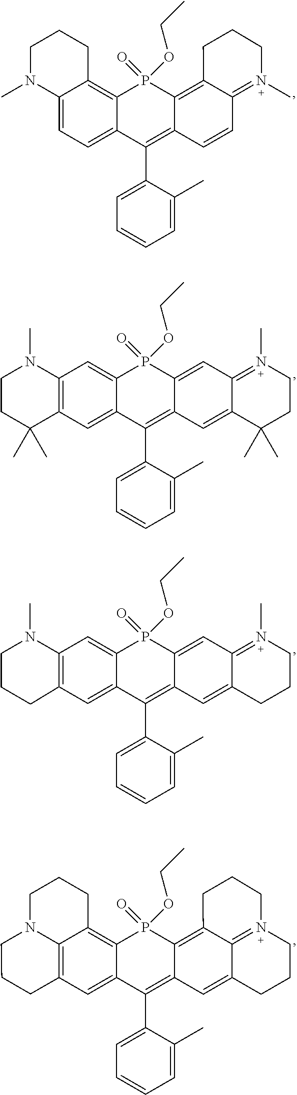

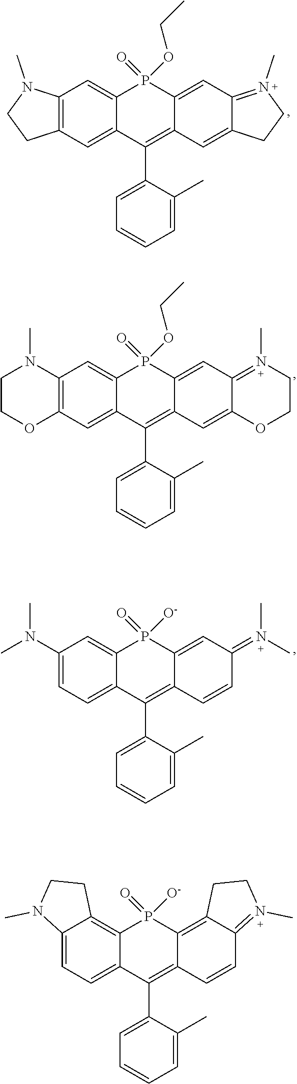

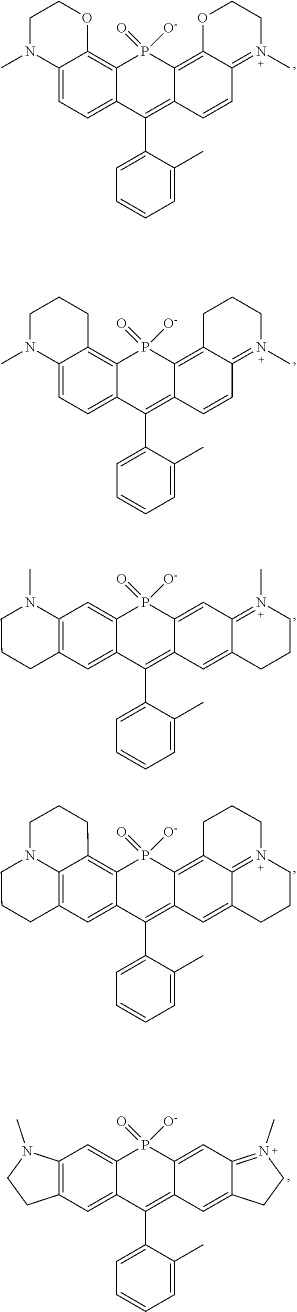

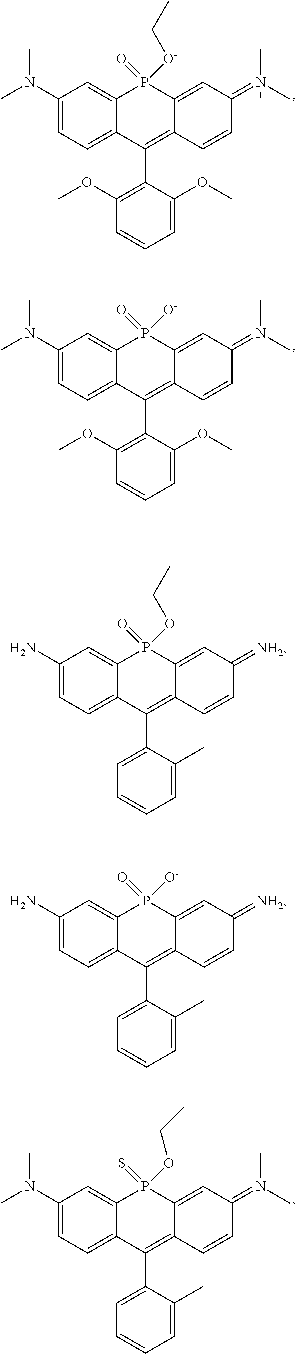

or any combination thereof.

In some aspects, the dye can have an excitation wavelength of form about 610 nm to about 1500 nm, or of about 610, 650, 700, 750, 800, 850, 900, 950, 1000, 1050, 1100, 1150, 1200, 1250, 1300, 1350, 1400, 1450, or about 1500 nm, or a combination of any of the foregoing values, or a range encompassing any of the foregoing values.

In another aspects, the dye can have an emission wavelength maximum of from about 630 nm to about 1600 nm, or of about 630, 650, 700, 750, 800, 850, 900, 950, 1000, 1050, 1100, 1150, 1200, 1250, 1300, 1350, 1400, 1450, 1500, 1550, or about 1600 nm, or a combination of any of the foregoing values, or a range encompassing any of the foregoing values.

In one aspect, the dye can have an half-life in water of from about 15 min to about 4 days, or of about 15, 30, or 45 minutes, or about 1, 2, 3, 4, 5, 6, 7, 8, 9, 10, 11, 12, 13, 14, 15, 16, 17, 18, 19, 20, 21, 22, or 23 hours, or 1, 1.5, 2, 2.5, 3, 3.5, or about 4 days, or a combination of any of the foregoing values, or a range encompassing any of the foregoing values.

In any of these aspects, the dye may not be quenched by glutathione.

In one aspect, the dye can have an extinction coefficient of from about 25,000 L/mol·cm to about 190,000 L/mol·cm, or of about 25,000, 30,000, 35,000, 40,000, 45,000, 50,000, 55,000, 60,000, 65,000, 70,000, 75,000, 80,000, 85,000, 90,000, 95,000, 100,000, 105,000, 110,000, 115,000, 120,000, 125,000, 130,000, 135,000, 140,000, 145,000, 150,000, 155,000, 160,000, 165,000, 170,000, 175,000, 180,000, 185,000, or about 190,000 L/mol·cm, or a combination of any of the foregoing values, or a range encompassing any of the foregoing values.

In some aspects, disclosed herein is a compound including the disclosed dye, wherein at least one instance of R₅ can be a substituted amide conjugated to a protein or peptide having affinity for a target molecule. In a further aspect, the compound can accumulate within tumors. In one aspect, the protein or peptide can be human serum albumin, an antibody, an antibody alternative, or a growth factor. As used herein, “antibody alternative” refers to a protein, peptide, nucleic acid aptamer, or other compound or construct that targets a specific protein of interest. In some aspects, an antibody alternative can include a monobody, a single chain variable fragment (scFv), or a designed ankyrin repeat protein (DARPin). Also disclosed is a method for imaging a tumor in a subject, the method including at least the steps of:
(a) administering a disclosed compound to the subject; and
(b) visualizing the tumor.

In one aspect, visualizing the tumor can be accomplished by fluorescence, Förster resonance energy transfer (FRET), fluorescence lifetime imaging, photoacoustic imaging, or any combination thereof.

In another aspect, disclosed herein is a dye having a structure of Formula II or an ionized form thereof:

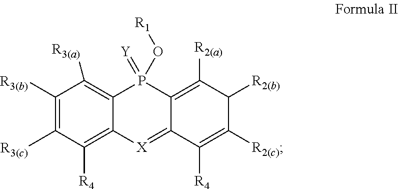

wherein R₁ is H, a C1-C6 linear or branched alkyl group, azide, alkyne, cyclooctyne, tetrazine, NHS ester, isocyanate, maleimide, iodoacetamide, diazonium salt, DNA-labeling agent, RNA-labeling agent, protein-labeling agent, or a 3-8-membered aryl, heteroaryl, cycloalkyl, or heterocycloalkyl group;
wherein R₂₍ₐ₎ is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{2(b)};
wherein R_{2(b)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, imine, azetidine, or sulfonate, or independently forms a 5- or 6-membered aryl, cycloalkyl, or heterocycloalkyl group with R₂₍ₐ₎ or R_{2(c)}, or forms 5- or 6-membered aryl, cycloalkyl, or heterocycloalkyl groups with both R₂₍ₐ₎ and R_{2(c)};
wherein R_{2(c)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{2(b)};
wherein R₃₍ₐ₎ is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, azetidine, or sulfonate, or forms a 5- or 6-membered aryl, cycloalkyl, or heterocycloalkyl group with R_{3(b)};
wherein R_{3(b)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or independently forms a 5- or 6-membered aryl, cycloalkyl, or heterocycloalkyl group with R₃₍ₐ₎ or R_{3(c)}, or forms 5- or 6-membered aryl, cycloalkyl, or heterocycloalkyl groups with both R₃₍ₐ₎ and R_{3(c)};
wherein R_{3(c)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{3(b)};
wherein each R₄ is, independently, selected from hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate;
wherein X is O, N, S, or CH; and
wherein Y is O or S.

In some aspects, the structure of Formula II or ionized form thereof can be selected from:

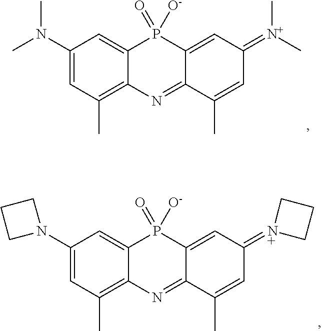

or any combination thereof.

In any of these aspects, the dye may not aggregate in solution. In one aspect, the dye displays a change in emission wavelength in aqueous solution relative to emission wavelength another solvent. In still another aspect, the dye can be biocompatible.

In one aspect, disclosed herein is a compound having a structure of Formula III or an ionized form thereof:

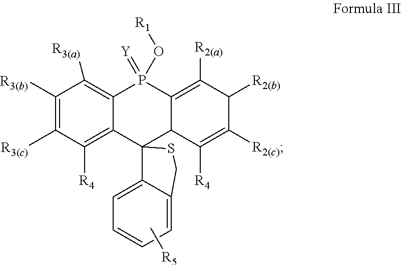

wherein R₁ is H, a C1-C6 linear or branched alkyl group, azide, alkyne, cyclooctyne, tetrazine, NHS ester, isocyanate, maleimide, iodoacetamide, diazonium salt, DNA-labeling agent, RNA-labeling agent, protein-labeling agent, or a 3-8-membered aryl, heteroaryl, cycloalkyl, or heterocycloalkyl group;
wherein R₂₍ₐ₎ is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{2(b)};
wherein R_{2(b)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, imine, or sulfonate, or independently forms a 5- or 6-membered aryl, cycloalkyl, or heterocycloalkyl group with R₂₍ₐ₎ or R_{2(c)}, or independently forms 5- or 6-membered aryl, cycloalkyl, or heterocycloalkyl groups with both R₂₍ₐ₎ and R_{2(c)};
wherein R_{2(c)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{2(b)};
wherein R₃₍ₐ₎ is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{3(b)};
wherein R_{3(b)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or independently forms a 5- or 6-membered aryl, cycloalkyl, or heterocycloalkyl group with R₃₍ₐ₎ or R_{3(c)}, or forms 5- or 6-membered aryl, cycloalkyl, or heterocycloalkyl groups with both R₃₍ₐ₎ and R_{3(c)};
wherein R_{3(c)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{3(b)};
wherein each R₄ is, independently, selected from hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate;
wherein each R₅ is, independently, selected from hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, C1-C4 alkoxy, amide, substituted amide, or sulfonate; and
wherein Y is O or S.

In another aspect, the compound can be:

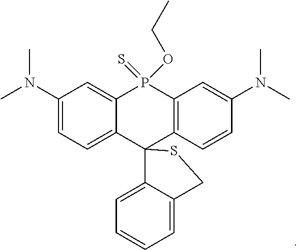

In still another aspect, disclosed herein is a method for making a dye, the method including the step of contacting a compound of Formula III with an oxidant including, but not limited to, hypochlorous acid (HOCl). Also disclosed are dyes made by the disclosed methods.

In one aspect, the dye can have the following structure:

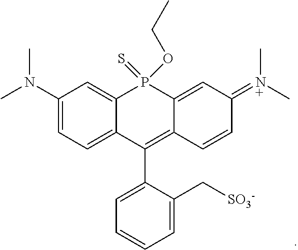

In another aspect, provided herein is a method for imaging a biological sample, the method including at least the steps of:
(a) contacting the biological sample with the compound, wherein the compound comprises a heterocyclic spiro ring, and wherein an oxidant in the biological sample opens the heterocyclic spiro ring, forming a dye; and
(b) imaging the biological sample.

In a further aspect, the oxidant can be HOCl. In some aspects, the HOCl is generated by the biological sample. In other aspects, the HOCl can be exogenously applied to the biological sample. In any of these aspects, the biological sample can be a cell, tissue, organ, organ system, or organism. In a further aspect, imaging the biological sample can be performed using photoacoustic imaging.

In still another aspect, disclosed herein is a method for detecting a disease associated with HOCl production in a subject, the method including imaging the subject using a disclosed method, wherein detection of a signal indicates the presence of the disease.

In another aspect, disclosed herein is a method for monitoring a disease associated with HOCl production in a subject, the method including at least the steps of:
(a) acquiring a first image of the subject at a first time using the disclosed method;
(b) acquiring a second image of the subject at a second time; and
(c) comparing the first image and the second image.

In a further aspect, the method is non-invasive. In one aspect, the disease can be acute myelogenous leukemia, rheumatoid arthritis, heart disease, heart attack, Parkinson's disease, or any combination thereof.

In one aspect, disclosed herein is a compound having a structure of Formula IV or an ionized form thereof:

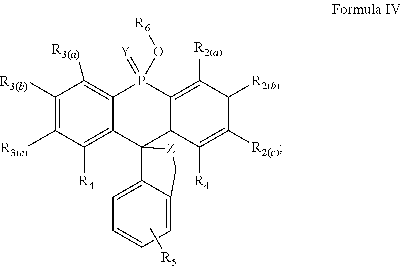

wherein R₂₍ₐ₎ is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{2(b)};
wherein R_{2(b)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, imine, or sulfonate, or independently forms a 5- or 6-membered aryl, cycloalkyl, or heterocycloalkyl group with R₂₍ₐ₎ or R_{2(c)}, or independently forms 5- or 6-membered aryl, cycloalkyl, or heterocycloalkyl groups with both R₂₍ₐ₎ and R_{2(c)};
wherein R_{2(c)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{2(b)};
wherein R₃₍ₐ₎ is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{3(b)};
wherein R_{3(b)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or independently forms a 5- or 6-membered aryl, cycloalkyl, or heterocycloalkyl group with R₃₍ₐ₎ or R_{3(c)}, or forms 5- or 6-membered aryl, cycloalkyl, or heterocycloalkyl groups with both R₃₍ₐ₎ and R_{3(c)};
wherein R_{3(c)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{3(b)};
wherein each R₄ is, independently, selected from hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate;
wherein each R₅ is, independently, selected from hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, C1-C4 alkoxy, amide, substituted amide, or sulfonate;
wherein Y is O or S;
wherein Z is O, S, substituted or unsubstituted amino, substituted or unsubstituted amide, substituted or unsubstituted alkyl, diazoketone, carboxyl, or a precursor thereof; and
wherein R₆ is a small molecule.

In one aspect, a “precursor” as used herein refers to a functional group that, upon exposure to at least one reactant (e.g., oxidant, reductant, or the like) solution condition (e.g., pH, salt concentration, solvent, temperature change, or the like), or external stimulus (e.g., irradiation) undergoes a chemical change to form the named functional group.

In some aspects, the small molecule can be coumarin, 1541B, doxorubicin, CA4, gemcitabine, camptothecin, another chemotherapeutic agent, another cytotoxic agent, an imaging probe, or any combination thereof. In one aspect, an imaging probe can include a DNA-labeling agent, an RNA-labeling agent, or a protein-labeling agent as disclosed herein. In one aspect, the compound can be

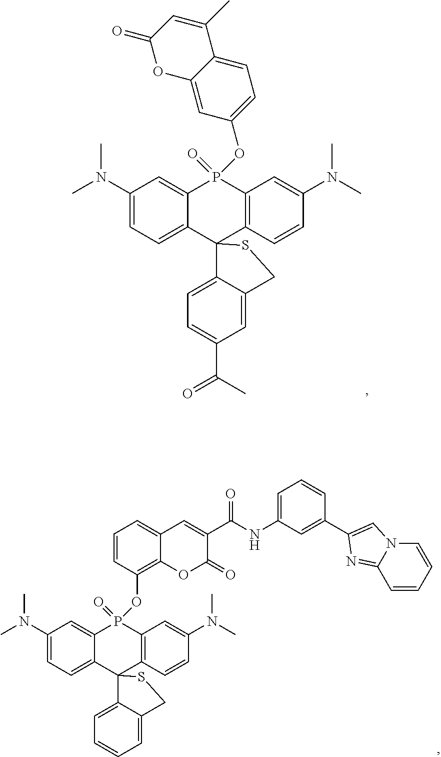

or any combination thereof

In any of these aspects, the compound may not be fluorescent.

In one aspect, disclosed herein is a method for simultaneously imaging a biological sample and delivering a small molecule to the biological sample, the method including at least the steps of:
(a) contacting the biological sample with a disclosed compound;
(b) triggering release of the small molecule from the compound; and
(c) imaging the biological sample.

In one aspect, triggering release of the small molecule can be accomplished by contacting the compound with an oxidant, contacting the compound with a reducing agent, exposing the compound to electromagnetic radiation, contacting the compound with a metal, contacting the compound with a reactive carbon species, exposing the compound to heat, exposing the compound to a change in pH, exposing the compound to a change in solvent polarity, exposing the compound to a change in local environment, contacting the compound with a target small molecule, or any combination thereof. In a further aspect, the metal can be sodium, potassium, magnesium, calcium, manganese, nickel, tungsten, iron, cobalt, copper, zinc, molybdenum, or any combination thereof. In one aspect, the oxidant is generated by the biological sample. In another aspect, the oxidant is exogenously applied to the biological sample. In any of these aspects, the oxidant can be HOCl, H₂O₂, superoxide, peroxides, nitric oxide, peroxynitrite, hydroxyl radical, another reactive oxygen species, a reactive nitrogen species, or any combination thereof. Reactive nitrogen species include, but are not limited to, nitroxyl anion, nitrosonium cation, higher oxides of nitrogen, S-nitrosothiols, dinitrosyl iron complexes, and related species. In an alternative aspect, the reducing agent can be generated by the biological sample or can be exogenously applied to the biological sample. In a further aspect, the reducing agent can be glutathione. In a still further aspect, the electromagnetic radiation can be ultraviolet light, visible light, or any combination thereof. In any of these aspects, the biological sample can be a cell, tissue, organ, organ system, or organism.

In still another aspect, in the disclosed method, following the release of the small molecule, the compound has a second structure having Formula V:

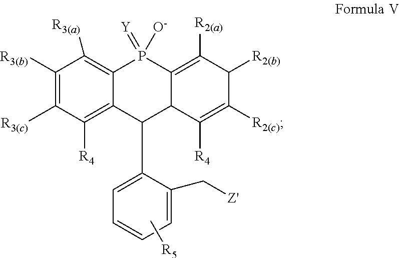

wherein R₂₍ₐ₎ is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{2(b)};
wherein R_{2(b)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, imine, or sulfonate, or independently forms a 5- or 6-membered aryl, cycloalkyl, or heterocycloalkyl group with R₂₍ₐ₎ or R_{2(c)}, or independently forms 5- or 6-membered aryl, cycloalkyl, or heterocycloalkyl groups with both R₂₍ₐ₎ and R_{2(c)};
wherein R_{2(c)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{2(b)};
wherein R₃₍ₐ₎ is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{3(b)};
wherein R_{3(b)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or independently forms a 5- or 6-membered aryl, cycloalkyl, or heterocycloalkyl group with R₃₍ₐ₎ or R_{3(c)}, or forms 5- or 6-membered aryl, cycloalkyl, or heterocycloalkyl groups with both R₃₍ₐ₎ and R_{3(c)};
wherein R_{3(c)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{3(b)};
wherein each R₄ is, independently, selected from hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate;
wherein each R₅ is, independently, selected from hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, C1-C4 alkoxy, amide, substituted amide, or sulfonate;
wherein Y is O or S; and
wherein Z′ is SO₃⁻, O⁻, substituted or unsubstituted amino, substituted or unsubstituted amide, substituted or unsubstituted alkyl, diazoketone, or carboxyl.

In a further aspect, the second structure can have the formula:

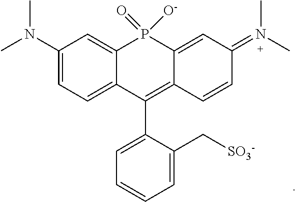

In one aspect, in the disclosed method, when the small molecule has been released, the small molecule, the second structure, or both, are fluorescent. In some aspects, the small molecule and the second structure have different maximum fluorescence emission wavelengths.

In any of these aspects, imaging the biological sample can be accomplished using confocal microscopy.

### Applications

### Labeling Reagents

In one aspect, the disclosed dyes described can be readily transformed into labeling reagents. One example of the synthesis of an NHS ester derivative of NR₆₆₆ is given in Scheme 1. Such a reagent can be utilized to label primary amino groups.

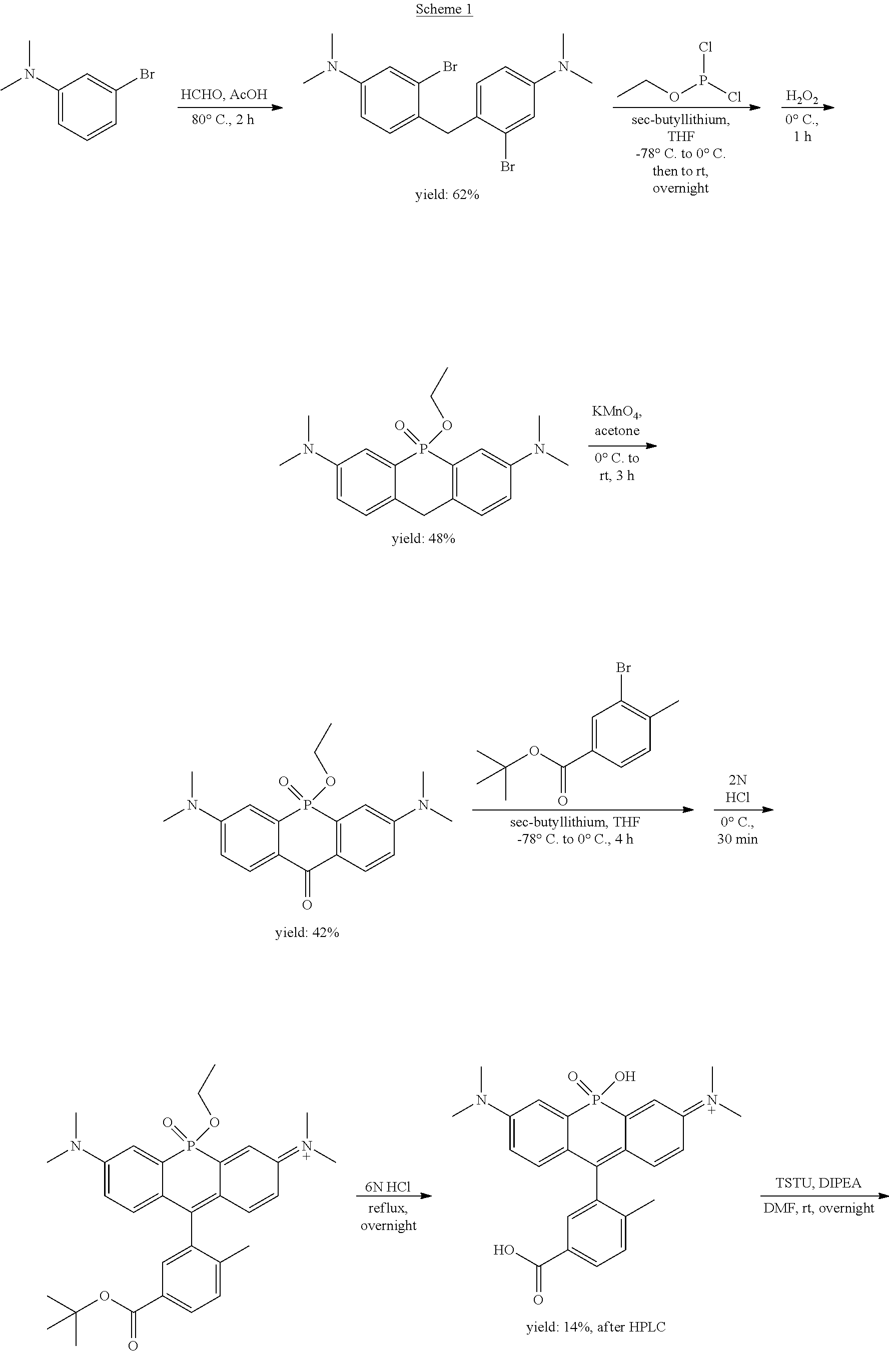

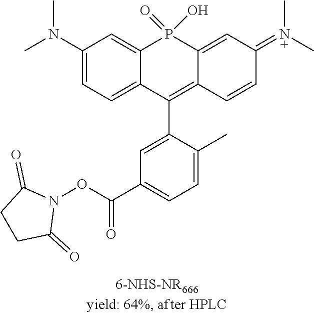

### Tuning Phosphinate Ester Stability

In one aspect, modifying the dye core or substitution on phosphorus can lead to substantial changes in phosphinate ester stability (see FIG. 1.1). In another aspect, influence of ether structure indicates that bulky substituents on the ester can also decrease hydrolysis rates, yielding stable phosphinate ester dyes for applications described herein (FIG. 1.3).

### Phosphinate Dyes as Theranostic Agents

In one aspect, disclosed herein is an approach in which the hydrolytic potential of phosphinate esters can be modulated by a target analyte (FIG. 1.4). Without wishing to be bound by theory, it is believed that analyte-induced changes in solubility can be used to modulate phosphinate ester hydrolysis. In a further aspect, such a system can harness the NR scaffold to respond to the altered biochemistry of a cell and simultaneously produce a fluorescent readout and deliver a small molecule agent.

### Phosphinate Dyes Produce a Photoacoustic Signal

Commercial photoacoustic tomography (PAT) systems rely on excitation of an optical absorber with pulsed laser light ranging from 680-980 nm. Subsequent dissipation of this excitation energy leads to local and rapid heating of the sample, resulting in thermoelastic expansion. The use of a pulsed excitation source yields fluctuating (megahertz) pressure waves that propagate through the sample as sound waves. Detection of these sound waves with a PAT instrument allows for reconstruction of a 3D image of the sample. Importantly, since PAT does not use ionizing radiation, it is potentially less hazardous than X-ray or positron emission tomography (PET). Since sound waves are diffracted to far lesser extent than light in biological samples, imaging depths of ˜10 cm can be achieved. Moreover, the resolution of PAT scales by ˜1/200^{th} of the imaging depth, meaning that a resolution of ˜350 μm can be obtained at 7 cm. However, there is a critical need within the field for the continued development of small molecules capable of absorbing within the 680-980 nm range of commercial PAT instrumentation. Disclosed herein are several NR dyes are capable of producing photoacoustic signal in commercially available PAT instrumentation (FIG. 1.5). In one aspect, the disclosed NR dyes fill a critical need, in this field, for small molecules capable of producing photoacoustic signal and represent a new class of dyes for use and further development as photoacoustic probes.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “an oxazine dye,” “a fluorophore,” or “a solvent,” includes, but is not limited to, mixtures or combinations of two or more such oxazine dyes, fluorophores, or solvents, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y.’ The range can also be expressed as an upper limit, e.g., ‘about x, y, z, or less' and should be interpreted to include the specific ranges of ‘about x,’ ‘about y,’ and ‘about z’ as well as the ranges of ‘less than x,’ less than y,’ and ‘less than z.’ Likewise, the phrase ‘about x, y, z, or greater’ should be interpreted to include the specific ranges of ‘about x,’ ‘about y,’ and ‘about z’ as well as the ranges of ‘greater than x,’ greater than y,’ and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e., one atmosphere).

“Quenching” as used herein refers to a decrease in absorbance or fluorescence emission of a dye due to a process acting on the dye or a chemical species present in a solution with the dye. For example, glutathione can cause quenching of some chromophores. In one aspect, the dyes disclosed herein do not exhibit quenching in the presence of glutathione.

“Biocompatible” as used herein refers to a compound, dye, or composition that does not damage or harm living tissue. In one aspect, a biocompatible material does not kill any living cells or trigger an immune response in a subject when the compound, dye, or composition is administered or applied to the subject.

“Aggregation” or “aggregate” as used herein refers to the tendency of a dye to form insoluble clusters or for individual dye molecules to associate with one another at increasing concentrations. In one aspect, thiazine dyes such as methylene blue tend to form aggregates, which can be visualized in absorption spectra as a blue-shifted shoulder that grows increasingly prominent with increased dye concentration (see FIG. 2.2D). In a further aspect, aggregates can be disrupted by addition of a surfactant to the dye solution. In one aspect, the disclosed dyes do not form aggregates or have a reduced tendency to form aggregates compared to parent molecules such as, for example, methylene blue. In one aspect, the reduction in aggregation or lack of aggregate forming can be visualized as absorption spectra that do not change with increasing dye concentration (when normalized for concentration) and/or that do not change with the addition of a surfactant such as, for example, sodium dodecyl sulfate (SDS) (see FIG. 2.2D).

In some aspects, an “oxazine-based” or “thiazine-based” dye, as used herein, refers to a synthetically modified or constructed molecule based on a known oxazine or thiazine dye, wherein any part of the molecule may retain the overall structure of the dye while eliminating one or more functional groups, such that an oxazine-based dye, for example, may no longer contain an oxazine moiety. In a further aspect, if the sulfur of a thiazine-based or the oxygen of an oxazine-based dye is substituted with another moiety, “thiazine-based” and “oxazine-based” can be used interchangeably herein.

As used herein, a “DNA-labeling agent” can include any small molecule useful for labeling or interacting with DNA including, but not limited to, an intercalating agent (e.g., ethidium bromide, proflavine, ellipticine, or the like), a minor groove binding molecule (e.g., Hoechst dyes such as Hoechst 33258 or Hoechst 33342, DAPI or 4′,6-diamidino-2-phenylindole), or a molecule that binds covalently to one or more components of the DNA (e.g., nucleobase, deoxyribose sugar, or phosphate backbone). In one aspect, a degree of overlap exists between DNA-labeling agents and “RNA-labeling agents,” although RNA-labeling agents can also include molecules that interact with tertiary RNA structures in conformations other than those typically adopted by DNA (e.g., double helix, packed chromatin structures, and the like). Exemplary DNA and RNA labeling agents include, but are not limited to: radionuclides (such as, for example, ³²P, ³⁵S, ³H, ¹⁴c, ¹²⁵I, ¹³¹I, and the like); chemiluminescent agents (such as, for example, acridinium esters, stabilized dioxetanes, and the like); quantum dots, metal nanoparticles (e.g., gold, silver, copper and platinum) or nanoclusters; enzymes (such as, for example, horseradish peroxidase, β-galactosidase, luciferase, alkaline phosphatase); colorimetric labels (such as, for example, dyes, colloidal gold, and the like); magnetic labels; and biotin, dioxigenin, haptens, and proteins for which antisera or monoclonal antibodies are available. In another aspect, the DNA- and/or RNA-labeling agent can be a fluorescent agent such as, for example, fluorescein and fluorescein dyes (e.g., fluorescein isothiocyanine or FITC, naphthofluorescein, 4′,5′-dichloro-2′,7-dimethoxy-fluorescein, 6-carboxyfluorescein or FAM), carbocyanine, merocyanine, styryl dyes, oxonol dyes, phycoerythrin, erythrosin, eosin, rhodamine dyes (e.g., carboxytetramethylrhodamine or TAMRA, carboxyrhodamine 60, carboxy-X-rhodamine (ROX), lissamine rhodamine B, rhodamine 6G, rhodamine Green, rhodamine Red, tetramethylrhodamine or TMR), coumarin and coumarin dyes (e.g., methoxycoumarin, dialkylaminocoumarin, hydroxycoumarin and aminomethylcoumarin or AMCA), Oregon Green Dyes (e.g., Oregon Green 488, Oregon Green 500, Oregon Green 514), Texas Red, Texas Red-X, Spectrum Red™, Spectrum Green™, cyanine dyes (e.g., Cy-3™, Cy-5™, Cy-3.5™, Cy-5.5™), Alexa Fluor dyes (e.g., Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), IRDyes (e.g., IRD40, IRD 700, IRD 800), or the like.

As used herein, a “protein-labeling agent” include any small molecule useful for labeling or interacting with proteins such as, for example a reactive dye, a reactive label, a reporter protein, or the like. In a further aspect, antibodies and antibody alternatives, binding pair members (e.g. biotin/avidin), and dyes providing optically detectable signals such as Coomassie Brilliant Blues R and G, silver staining, Sypro Ruby, Sypro Orange, Eosin, nitro blue terrazolium, Deep Purple, Amido Black 10B, Coomassie Violet, Brilliant Sulfoflavine, Hungarian Red, Tartrazine, Patent Blue VF, Crocein Scarlet, Lissamine Green, Sulforhodamine B, pyranine, Ponceau S, another fluorescent dye, or any combination thereof. In a further aspect, numerous DNA- and RNA-labeling agents listed herein also function as protein-labeling agents.

As used herein, “anti-cancer agents” include, but are not limited to, epirubicin, paclitaxel, doxorubicin, gemcitabine, cisplatin, methotrexate, 5-fluorouracil, betulinic acid, amphotericin B, diazepam, nystatin, docetaxel, a maytansinoid, a PD-1 inhibitor, a protein kinase inhibitor, a P-glycoprotein inhibitor, an autophage inhibitor, a PARP inhibitor, an aromatase inhibitor, a monoclonal antibody, a photosensitizer, a radiosensitizer, an interleukin, an antiandrogen, or any combination thereof.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g., fluorine, chlorine, bromine, or iodine. The term “polyhaloalkyl” specifically refers to an alkyl group that is independently substituted with two or more halides, i.e., each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “alkanediyl” as used herein, refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, and —CH₂CH₂CH₂— are non-limiting examples of alkanediyl groups.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a shorthand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) and —N(-alkyl)₂, where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen” or “halide,” as used herein can be used interchangeably and refer to F, Cl, Br, or I.

The terms “pseudohalide,” “pseudohalogen” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized, and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxy” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a shorthand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” . . . “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

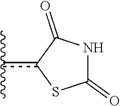

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, sulfur, fluorine and chlorine, such as ²H, ³H, ¹³C, ¹⁴C ¹⁵N, ¹⁸O, ¹⁷O ³⁵S, ¹⁸F, and ³⁶Cl, respectively. Compounds further comprise prodrugs thereof and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g., “Crystal Engineering of the Composition of Pharmaceutical Phases. Do Pharmaceutical Co-crystals Represent a New Path to Improved Medicines?” Almarasson, O., et al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

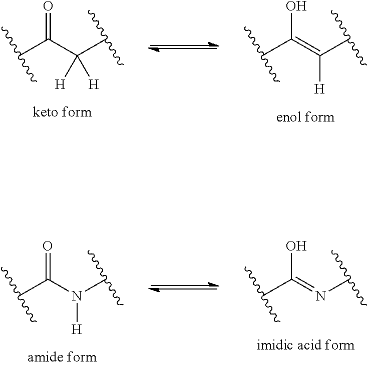 [SMILES: *CC(*)=O ; */C=C(/*)O ; *NC(*)=O ; */N=C(/*)O]

Likewise, amides with an N-hydrogen can exist in an equilibrium of the amide form and the imidic acid form. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

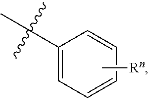

which is understood to be equivalent to a formula:

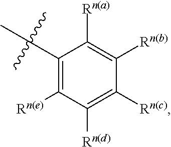

wherein n is typically an integer. That is, Rⁿ is understood to represent five independent substituents, R^{n(a)}, R^{n(b)}, R^{n(c)}, R^{n(d)}, and R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

As used herein, “ionized form” refers to a form of a compound displaying one or more charges in any combination (for example, a single positive charge, or both a positive and a negative charge on different atoms, respectively, or the like). In some aspects, an ionized form of a compound can be permanent, e.g., that ionized form resulting from a nitrogen atom participating in four covalent bonds. In other aspects, the ionized form of the compound can be transient, e.g., the compound may gain or lose a proton depending on solvent conditions, solution pH, or the pKₐ of a chemical group, such as when the compound possesses a carboxylic acid or a phosphate group or the like. In any of these aspects, the ionized form of the compound can be associated with a counter ion.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### ASPECTS

The present disclosure can be described in accordance with the following numbered aspects, which should not be confused with the claims.

Aspect 1. A dye having a structure of Formula I or an ionized form thereof:

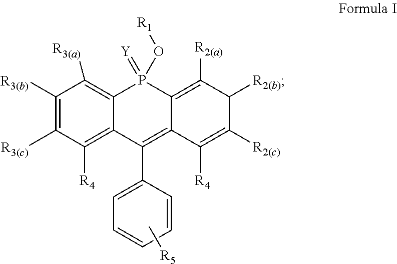

wherein R₁ is H, a C1-C6 linear or branched alkyl group, azide, alkyne, cyclooctyne, tetrazine, N-hydroxysuccinimide (NHS) ester, isocyanate, maleimide, iodoacetamide, diazonium salt, DNA-labeling agent, RNA-labeling agent, protein-labeling agent, or a 3-8-membered aryl, heteroaryl, cycloalkyl, or heterocycloalkyl group;
wherein R₂₍ₐ₎ is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{2(b)};
wherein R_{2(b)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, imine, or sulfonate, or independently forms a 5- or 6-membered aryl, cycloalkyl, or heterocycloalkyl group with R₂₍ₐ₎ or R_{2(c)}, or independently forms 5- or 6-membered aryl, cycloalkyl, or heterocycloalkyl groups with both R₂₍ₐ₎ and R_{2(c)};
wherein R_{2(c)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{2(b)};
wherein R₃₍ₐ₎ is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{3(b)};
wherein R_{3(b)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or independently forms a 5- or 6-membered aryl, cycloalkyl, or heterocycloalkyl group with R₃₍ₐ₎ or R_{3(c)}, or forms 5- or 6-membered aryl, cycloalkyl, or heterocycloalkyl groups with both R₃₍ₐ₎ and R_{3(c)};
wherein R_{3(c)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{3(b)};
wherein each R₄ is, independently, selected from hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate;
wherein each R₅ is, independently, selected from hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, C1-C4 alkoxy, amide, substituted amide, or sulfonate; and
wherein Y is O or S.

Aspect 2. The dye of aspect 1, wherein the structure of Formula I or ionized form thereof is selected from:

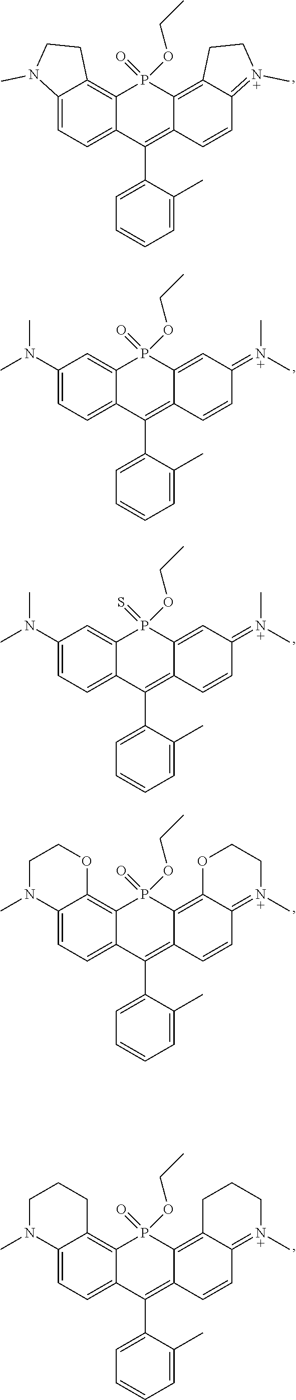

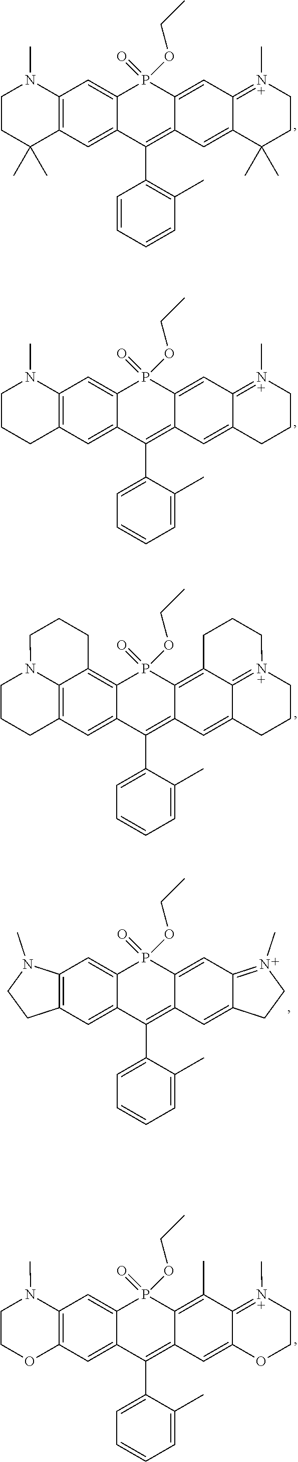

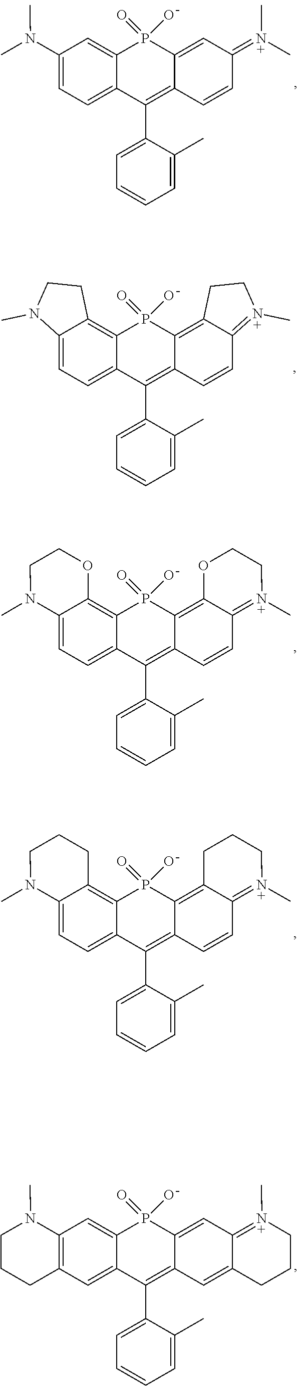

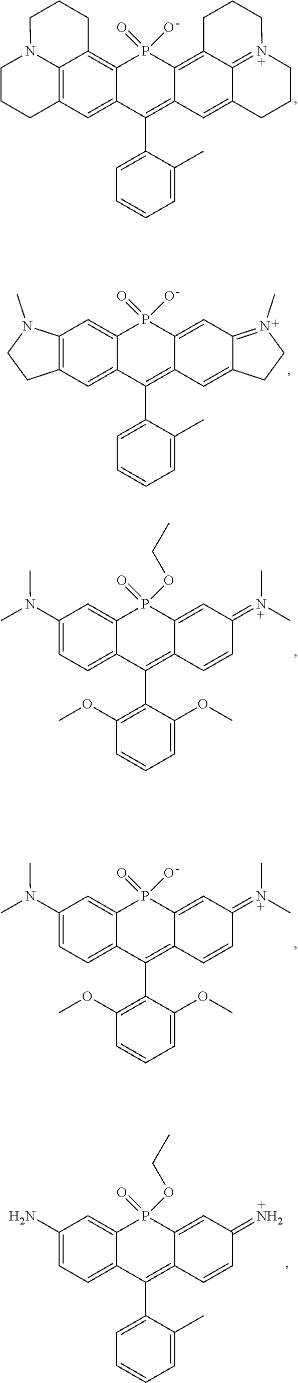

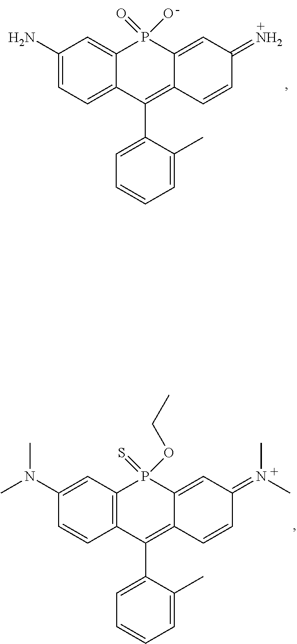

or any combination thereof.

Aspect 3. The dye of aspect 1 or 2, wherein the dye has an excitation wavelength of from about 610 nm to about 1600 nm.

Aspect 4. The dye of any one of aspects 1-3, wherein the dye has an emission wavelength maximum of from about 630 nm to about 1600 nm.

Aspect 5. The dye of any one of aspects 1-4, wherein the dye has a half-life in water of from about 15 min to about 4 days.

Aspect 6. The dye of any one of aspects 1-5, wherein the dye is not quenched by glutathione.

Aspect 7. The dye of any one of aspects 1-6, wherein the dye has an extinction coefficient of from about 25,000 L/mol·cm to about 190,000 L/mol·cm.

Aspect 8. A compound comprising the dye of any one of aspects 1-7, wherein at least one instance of R₅ comprises a substituted amide conjugated to a protein or peptide having affinity for a target molecule.

Aspect 9. The compound of aspect 8, wherein the compound accumulates within tumors.

Aspect 10. The compound of aspect 8 or 9, wherein the protein is human serum albumin, an antibody, an antibody alternative, or a growth factor.

Aspect 11. A method for imaging a tumor in a subject, the method comprising:
(a) administering the compound of any one of aspects 8-10 to the subject; and
(b) visualizing the tumor.

Aspect 12. The method of aspect 10, wherein visualizing the tumor comprises fluorescence, Förster resonance energy transfer (FRET), fluorescence lifetime imaging, photoacoustic imaging, or any combination thereof.

Aspect 13. A dye having a structure of Formula II or an ionized form thereof:

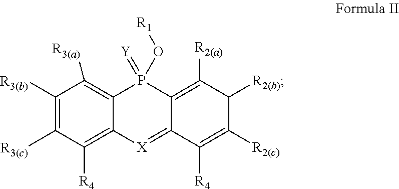

wherein R₁ is H, a C1-C6 linear or branched alkyl group, azide, alkyne, cyclooctyne, tetrazine, NHS ester, isocyanate, maleimide, iodoacetamide, diazonium salt, DNA-labeling agent, RNA-labeling agent, protein-labeling agent, or a 3-8-membered aryl, heteroaryl, cycloalkyl, or heterocycloalkyl group;
wherein R₂₍ₐ₎ is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{2(b)};
wherein R_{2(b)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, imine, azetidine, or sulfonate, or independently forms a 5- or 6-membered aryl, cycloalkyl, or heterocycloalkyl group with R₂₍ₐ₎ or R_{2(c)}, or forms 5- or 6-membered aryl, cycloalkyl, or heterocycloalkyl groups with both R₂₍ₐ₎ and R_{2(c)};
wherein R_{2(c)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{2(b)};
wherein R₃₍ₐ₎ is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, azetidine, or sulfonate, or forms a 5- or 6-membered aryl, cycloalkyl, or heterocycloalkyl group with R_{3(b)};
wherein R_{3(b)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or independently forms a 5- or 6-membered aryl, cycloalkyl, or heterocycloalkyl group with R₃₍ₐ₎ or R_{3(c)}, or forms 5- or 6-membered aryl, cycloalkyl, or heterocycloalkyl groups with both R₃₍ₐ₎ and R_{3(c)};
wherein R_{3(c)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{3(b)};
wherein each R₄ is, independently, selected from hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate;
wherein X is O, N, S, or CH; and
wherein Y is O or S.

Aspect 14. The dye of aspect 13, wherein the structure of Formula II or ionized form thereof is selected from:

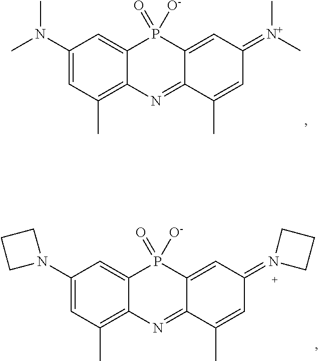

or any combination thereof.

Aspect 15. The dye of aspect 13 or 14, wherein the dye does not aggregate in solution.

Aspect 16. The dye of any one of aspects 13-15, wherein the dye displays a change in emission wavelength in aqueous solution relative to emission wavelength another solvent.

Aspect 17. The dye of any one of aspects 1-7 or 13-16, wherein the dye is biocompatible.

Aspect 18. A compound having a structure of Formula III or an ionized form thereof:

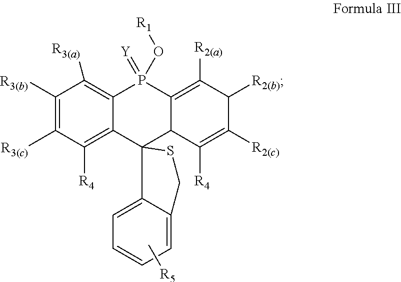

wherein R₁ is H, a C1-C6 linear or branched alkyl group, azide, alkyne, cyclooctyne, tetrazine, NHS ester, isocyanate, maleimide, iodoacetamide, diazonium salt, DNA-labeling agent, RNA-labeling agent, protein-labeling agent, or a 3-8-membered aryl, heteroaryl, cycloalkyl, or heterocycloalkyl group;
wherein R₂₍ₐ₎ is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{2(b)};
wherein R_{2(b)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, imine, or sulfonate, or independently forms a 5- or 6-membered aryl, cycloalkyl, or heterocycloalkyl group with R₂₍ₐ₎ or R_{2(c)}, or independently forms 5- or 6-membered aryl, cycloalkyl, or heterocycloalkyl groups with both R₂₍ₐ₎ and R_{2(c)};
wherein R_{2(c)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{2(b)};
wherein R₃₍ₐ₎ is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{3(b)};
wherein R_{3(b)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or independently forms a 5- or 6-membered aryl, cycloalkyl, or heterocycloalkyl group with R₃₍ₐ₎ or R_{3(c)}, or forms 5- or 6-membered aryl, cycloalkyl, or heterocycloalkyl groups with both R₃₍ₐ₎ and R_{3(c)};
wherein R_{3(c)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{3(b)};
wherein each R₄ is, independently, selected from hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate;
wherein each R₅ is, independently, selected from hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, C1-C4 alkoxy, amide, substituted amide, or sulfonate; and
wherein Y is O or S.

Aspect 19. The compound of aspect 18, wherein the compound comprises:

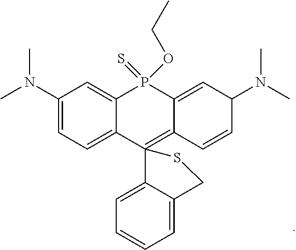

Aspect 20. A method for making a dye, the method comprising contacting the compound of aspect 18 or 19 with an oxidant.

Aspect 21. The method of aspect 20, wherein the oxidant comprises hypochlorous acid (HOCl).

Aspect 22. A dye made by the method of aspect 20 or 21.

Aspect 23. The dye of aspect 22, wherein the dye comprises:

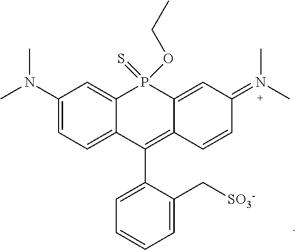

Aspect 24. A method for imaging a biological sample, the method comprising:
(a) contacting the biological sample with the compound of aspect 18 or 19, wherein the compound comprises a heterocyclic spiro ring, and wherein an oxidant in the biological sample opens the heterocyclic spiro ring, forming a dye; and
(b) imaging the biological sample.

Aspect 25. The method of aspect 24, wherein the oxidant comprises HOCl.

Aspect 26. The method of aspect 24 or 25, wherein the HOCl is generated by the biological sample.

Aspect 27. The method of aspect 24 or 25, wherein the HOCl is exogenously applied to the biological sample.

Aspect 28. The method of any one of aspects 24-27, wherein the biological sample comprises a cell, tissue, organ, organ system, or organism.

Aspect 29. The method of any one of aspects 24-28, wherein imaging the biological sample comprises photoacoustic imaging.

Aspect 30. A method for detecting a disease associated with HOCl production in a subject, the method comprising imaging the subject using the method of any one of aspects 24-29, wherein detection of a signal indicates the presence of the disease.

Aspect 31. A method for monitoring a disease associated with HOCl production in a subject, the method comprising:
(a) acquiring a first image of the subject at a first time using the method of any one of aspects 24-29;
(b) acquiring a second image of the subject at a second time; and
(c) comparing the first image and the second image.

Aspect 32. The method of aspect 31, wherein the method is non-invasive.

Aspect 33. The method of any one of aspects 30-32, wherein the disease comprises acute myelogenous leukemia, rheumatoid arthritis, heart disease, heart attack, Parkinson's disease, or any combination thereof.

Aspect 34. A compound having a structure of Formula IV or an ionized form thereof:

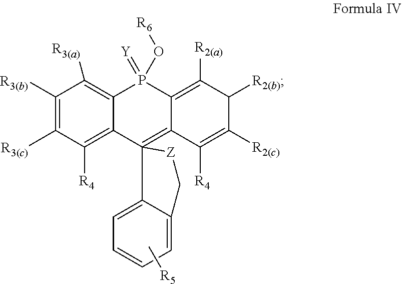

wherein R₂₍ₐ₎ is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{2(b)};
wherein R_{2(b)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, imine, or sulfonate, or independently forms a 5- or 6-membered aryl, cycloalkyl, or heterocycloalkyl group with R₂₍ₐ₎ or R_{2(c)}, or independently forms 5- or 6-membered aryl, cycloalkyl, or heterocycloalkyl groups with both R₂₍ₐ₎ and R_{2(c)};
wherein R_{2(c)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{2(b)};
wherein R₃₍ₐ₎ is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{3(b)};
wherein R_{3(b)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or independently forms a 5- or 6-membered aryl, cycloalkyl, or heterocycloalkyl group with R₃₍ₐ₎ or R_{3(c)}, or forms 5- or 6-membered aryl, cycloalkyl, or heterocycloalkyl groups with both R₃₍ₐ₎ and R_{3(c)};
wherein R_{3(c)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{3(b)};
wherein each R₄ is, independently, selected from hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate;
wherein each R₅ is, independently, selected from hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, C1-C4 alkoxy, amide, substituted amide, or sulfonate;
wherein Y is O or S;
wherein Z is O, S, substituted or unsubstituted amino, substituted or unsubstituted amide, substituted or unsubstituted alkyl, diazoketone, carboxyl, or a precursor thereof; and
wherein R₆ comprises a small molecule.

Aspect 35. The compound of aspect 34, wherein the small molecule comprises coumarin, 1541B, doxorubicin, CA4, gemcitabine, camptothecin, another chemotherapeutic agent, another cytotoxic agent, an imaging probe, or any combination thereof.

Aspect 36. The compound of aspect 34 or 35, wherein the compound comprises

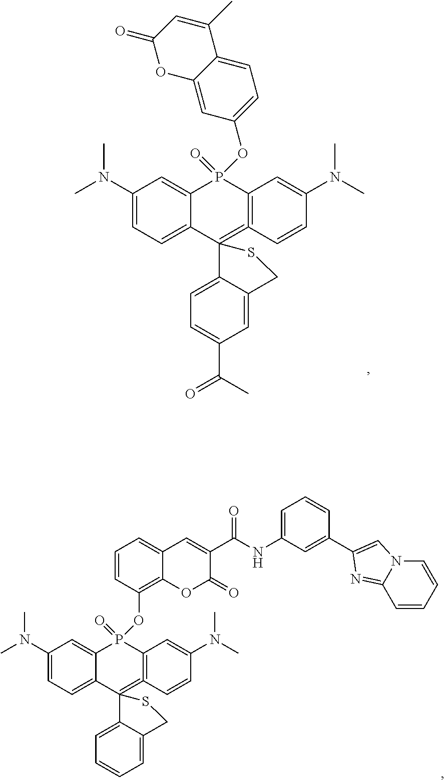

or any combination thereof.

Aspect 37. The compound of any one of aspects 34-36, wherein the compound is not fluorescent.

Aspect 38. A method for simultaneously imaging a biological sample and delivering a small molecule to the biological sample, the method comprising:
(a) contacting the biological sample with the compound of any one of aspects 34-37;
(b) triggering release of the small molecule from the compound; and
(c) imaging the biological sample.

Aspect 39. The method of aspect 38, wherein triggering release of the small molecule comprises contacting the compound with an oxidant, contacting the compound with a reducing agent, exposing the compound to electromagnetic radiation, contacting the compound with a metal, contacting the compound with a reactive carbon species, exposing the compound to heat, exposing the compound to a change in pH, exposing the compound to a change in solvent polarity, exposing the compound to a change in local environment, contacting the compound with a target small molecule, or any combination thereof.

Aspect 40. The method of aspect 39, wherein the oxidant is generated by the biological sample.

Aspect 41. The method of aspect 39, wherein the oxidant is exogenously applied to the biological sample.

Aspect 42. The method of any one of aspects 39-41, wherein the oxidant comprises HOCl, H₂O₂, superoxide, peroxides, nitric oxide, peroxynitrite, hydroxyl radical, another reactive oxygen species, a reactive nitrogen species, or any combination thereof.

Aspect 43. The method of aspect 39, wherein the reducing agent is generated by the biological sample.

Aspect 44. The method of aspect 39 or 43, wherein the reducing agent is exogenously applied to the biological sample.

Aspect 45. The method of any one of aspects 39, 43, or 44, wherein the reducing agent comprises glutathione.

Aspect 46. The method of aspect 39, wherein the electromagnetic radiation comprises ultraviolet light, visible light, or any combination thereof.

Aspect 47. The method of any one of aspects 38-46, wherein the biological sample comprises a cell, tissue, organ, organ system, or organism.

Aspect 48. The method of any one of aspects 38-47, wherein, following release of the small molecule, the compound has a second structure having Formula V:

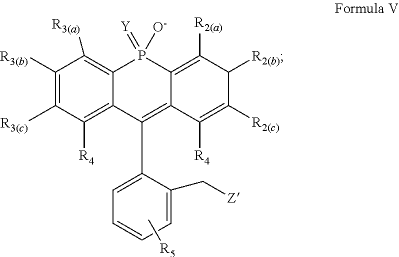

wherein R₂₍ₐ₎ is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{2(b)};
wherein R_{2(b)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, imine, or sulfonate, or independently forms a 5- or 6-membered aryl, cycloalkyl, or heterocycloalkyl group with R₂₍ₐ₎ or R_{2(c)}, or independently forms 5- or 6-membered aryl, cycloalkyl, or heterocycloalkyl groups with both R₂₍ₐ₎ and R_{2(c)};
wherein R_{2(c)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{2(b)};
wherein R₃₍ₐ₎ is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{3(b)};
wherein R_{3(b)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or independently forms a 5- or 6-membered aryl, cycloalkyl, or heterocycloalkyl group with R₃₍ₐ₎ or R_{3(c)}, or forms 5- or 6-membered aryl, cycloalkyl, or heterocycloalkyl groups with both R₃₍ₐ₎ and R_{3(c)};
wherein R_{3(c)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{3(b)};
wherein each R₄ is, independently, selected from hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate;
wherein each R₅ is, independently, selected from hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, C1-C4 alkoxy, amide, substituted amide, or sulfonate;
wherein Y is O or S; and
wherein Z′ is SO₃—, O—, substituted or unsubstituted amino, substituted or unsubstituted amide, substituted or unsubstituted alkyl, diazoketone, or carboxyl.

Aspect 49. The method of aspect 48, wherein the second structure has the formula

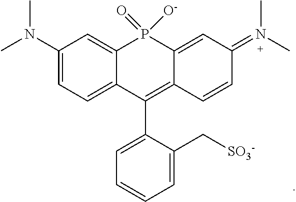

Aspect 50. The method of aspect 48 or 49, wherein, when the small molecule has been released, the small molecule, the second structure, or both are fluorescent.

Aspect 51. The method of aspect 50, wherein the small molecule and the second structure have different maximum fluorescence emission wavelengths.

Aspect 52. The method of any one of aspects 38-51, wherein imaging the biological sample comprises confocal microscopy.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Compound Synthesis and Characterization

### General Synthetic Procedure A

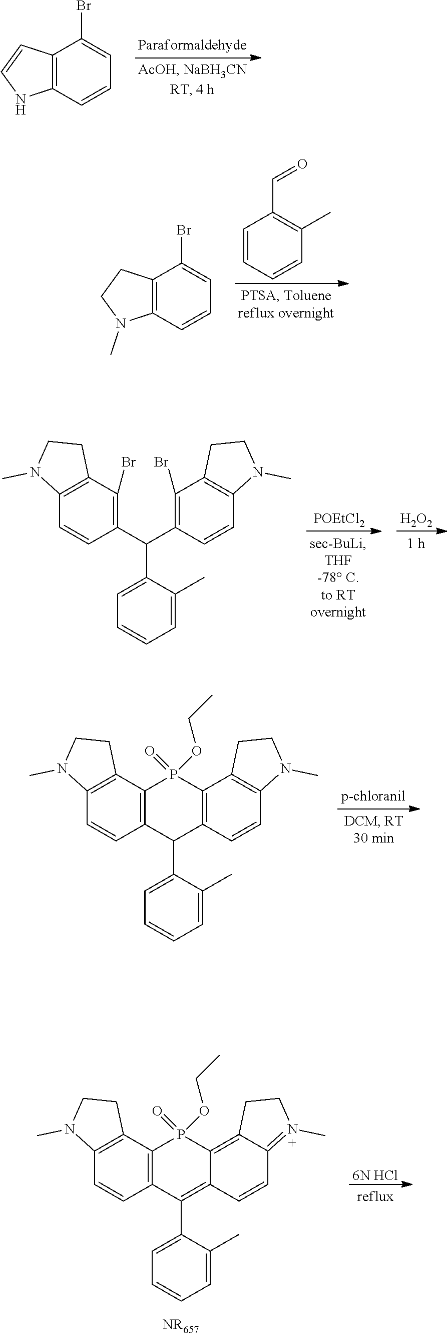

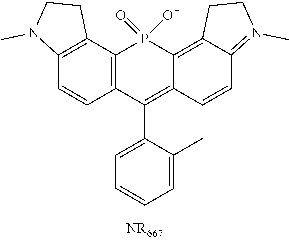

### 4-bromo-1-methylindoline

4-Bromoindole (10 g, 51 mmol) and paraformaldehyde (15.3 g, 510 mmol) were stirred in acetic acid (250 mL) and the temperature was lowered to 10˜15° C. Sodium cyanoborohydride (16 g, 255 mmol) was then added slowly over 30 mm and the reaction was stirred at room temperature for an additional 4 h. The reaction was neutralized with NaOH at 0° C. and extracted with dichloromethane (DCM). Flash column chromatography (10˜30% ethyl acetate/hexane) gave 7 g product in 65% yield.

¹H NMR (300 MHz, Chloroform-d) δ 6.96 (t, J=7.9 Hz, 1H), 6.80 (d, J=8.0 Hz, 1H), 6.39 (d, J=7.7 Hz, 1H), 3.39 (t, J=8.3 Hz, 2H), 2.99 (t, J=8.3 Hz, 2H), 2.78 (s, 3H). ¹³C NMR (75 MHz, Chloroform-d) δ 154.40, 130.42, 129.06, 120.31, 119.41, 105.47, 54.94, 35.99, 29.95. MS (ESI) m/z calculated for C₉H₁₀BrN [M+H]⁺ 213.1, found 213.2

### 5,5′-(o-tolylmethylene)bis(4-bromo-1-methylindoline)

4-bromo-1-methylindoline (6.36 g, 30 mmol) and 2-methylbenzaldehyde (1.155 mL, 10 mmol) were dissolved in toluene (40 mL). p-Toluenesulfonic acid monohydrate (1.9 g, 10 mmol) was then added and the solution was refluxed at 130° C. overnight using a Dean-Stark apparatus. Extra toluene was removed by evaporation and the resulting oil mixture was dissolved in DCM and extracted using saturated sodium bicarbonate solution. The DCM layer was dried over sodium sulfate, filtered, and the solvent was evaporated. Flash column chromatography (10˜70% DCM/hexane) was performed to give the product as a white solid (2.42 g, 46%).

¹H NMR (300 MHz, Chloroform-d) δ 7.21-7.01 (m, 3H), 6.73 (d, J=7.2 Hz, 1H), 6.48 (d, J=8.0 Hz, 2H), 6.24 (d, J=8.0 Hz, 2H), 5.96 (s, 1H), 3.36 (t, J=8.3 Hz, 4H), 2.97 (t, J=8.2 Hz, 4H), 2.72 (s, 6H), 2.21 (s, 3H). ¹³C NMR (75 MHz, Chloroform-d) δ 152.70, 142.10, 137.12, 131.57, 131.09, 130.26, 130.04, 128.89, 126.24, 125.62, 122.86, 105.19, 55.26, 51.35, 36.24, 31.03, 19.73. MS (ESI) m/z calculated for C₂₆H₂₆Br₂N₂[M+H]⁺ 527.3, found 527.4.

### NR₆₉₇

5,5′-(o-tolylmethylene)bis(4-bromo-1-methylindoline) (1.053 g, 2 mmol) was dissolved in anhydrous THF and diethyl ether mixture (25 mL+25 mL) and the reaction temperature was lowered to −78° C. sec-Butyllithium (1.4 M in cyclohexane, 3.14 mL, 4.4 mmol) was added dropwise over 10 min and the resulting mixture was further stirred for 1 hr at the same temperature followed by dropwise addition of ethyl dichlorophosphite (163 μL, 2.2 mmol) over 20 min. The reaction mixture was maintained at −78° C. for another 3 h and the temperature was slowly raised to room temperature and left overnight. The reaction was lowered to 0° C. and hydrogen peroxide (50 wt. % in H₂O, 2 mL) was added dropwise to the mixture, which was stirred for 1 h on ice. The reaction was then quenched by saturated sodium sulfite solution and extracted using DCM. The organic layer was collected, dried over sodium sulfate, and the solvent was removed. The resulting yellow solid was dissolved in DCM (10 mL) and p-chloranil (983 mg, 4 mmol) was added. The mixture was stirred for 30 min at room temperature and then the temperature was lowered to 0° C. The solid was filtered and washed using cold DCM (2×10 mL). The solvent was removed, and a short silica 60 column was performed to remove extra p-chloranil (0˜20% methanol/DCM with 0.1% acetic acid). After concentration, the dark green oil was dissolved in HPLC buffer (50% acetonitrile in water with 0.1% trifluoroacetic acid) and clarified by centrifugation. The product peak from HPLC was lyophilized to yield a dark green solid (232 mg, 20.3%).

¹H NMR (300 MHz, Chloroform-d) δ 7.53-7.41 (m, 1H), 7.36 (t, J=6.8 Hz, 2H), 7.11 (d, J=7.8 Hz, 1H), 6.99 (dd, J=9.1, 5.9 Hz, 2H), 6.44 (d, J=9.2 Hz, 2H), 4.24-3.74 (m, 8H), 3.47 (ddt, J=18.4, 8.4, 3.9 Hz, 2H), 3.25 (s, 6H), 2.00 (s, 3H), 1.33 (t, J=7.1 Hz, 3H). ¹³C NMR (75 MHz, Chloroform-d) δ 160.67, 160.56, 158.63, 158.46, 142.75, 142.63, 141.32, 141.26, 136.69, 136.07, 130.40, 129.37, 128.93, 126.90, 126.05, 125.27, 125.24, 125.13, 108.61, 62.98, 62.89, 55.87, 33.91, 27.06, 27.00, 19.25, 16.32, 16.22. ³¹P NMR (121 MHz, Chloroform-d) δ 14.04. MS (ESI) m/z calculated for C₃₀H₃₀F₃N₂O₄P [M-CF₃COO]⁺457.5, found 457.5

### NR₆₆₇

NR₆₉₇ (100 mg, 0.175 mmol) was dissolved in HCl (6 N, 10 mL) and the solution was refluxed overnight. After evaporating the solvent, the resulting blue solid was purified by HPLC, yielding a dark blue solid (54 mg, 72%).

¹H NMR (300 MHz, DMSO-d₆) δ 7.43 (dq, J=15.1, 7.5 Hz, 3H), 7.12 (d, J=7.3 Hz, 1H), 6.77 (dd, J=9.0, 5.4 Hz, 2H), 6.66 (d, J=9.2 Hz, 2H), 3.99 (t, J=6.8 Hz, 4H), 3.44 (t, J=6.0 Hz, 4H), 3.16 (s, 6H), 1.99 (s, 3H). ¹³C NMR (75 MHz, DMSO-d₆) δ 158.69, 158.54, 141.09, 140.98, 139.50, 139.44, 137.94, 135.91, 130.70, 129.53, 129.36, 126.41, 124.13, 124.02, 108.85, 55.78, 34.04, 26.71, 26.65, 19.24. ³¹P NMR (121 MHz, DMSO-d₆) δ 4.99. MS (ESI) m/z calculated for C₂₆H₂₅N₂O₂P [M+H]⁺ 429.5, found 429.6.

Other NR derivatives were synthesized following general synthetic procedure A.

### NR₇₇₃

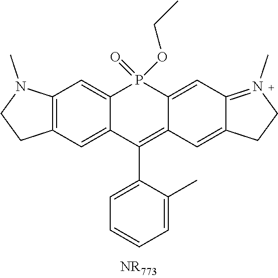

Green solid. ¹H NMR (300 MHz, Chloroform-d) δ 7.53-7.44 (m, 1H), 7.39 (dd, J=13.0, 5.2 Hz, 3H), 7.31 (d, J=3.5 Hz, 1H), 7.09 (dd, J=21.2, 7.5 Hz, 1H), 6.62 (d, J=6.0 Hz, 2H), 4.05 (ddt, J=36.1, 15.6, 7.6 Hz, 6H), 3.32 (s, 6H), 3.05 (t, J=8.3 Hz, 4H), 2.06 (d, J=23.0 Hz, 3H), 1.34 (t, J=7.0 Hz, 3H). ¹³C NMR (75 MHz, Chloroform-d) δ 161.61, 160.66, 160.18, 158.03, 157.84, 138.62, 136.98, 136.51, 135.74, 134.74, 134.71, 131.84, 131.69, 130.82, 130.54, 129.46, 129.14, 128.42, 126.30, 125.39, 125.27, 118.11, 112.15, 112.08, 62.19, 62.10, 55.36, 34.15, 26.34, 19.44, 16.34, 16.25. ³¹P NMR (121 MHz, Chloroform-d) δ 14.07, 13.99. MS (ESI) m/z calculated for C₃₀H₃₀F₃N₂O₄P [M-CF₃COO]⁺457.5, found 457.5.

### NR₇₂₁

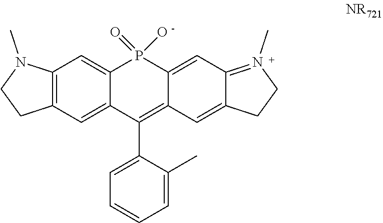

Green solid. ¹H NMR (300 MHz, Chloroform-d) δ 7.66-7.31 (m, 5H), 7.11 (d, J=7.3 Hz, 1H), 6.56 (d, J=4.5 Hz, 2H), 3.82 (t, J=8.0 Hz, 4H), 3.24 (s, 6H), 2.96 (t, J=8.0 Hz, 4H), 2.08 (s, 3H). ¹³C NMR (75 MHz, Chloroform-d) δ 162.74, 162.62, 158.58, 158.40, 148.57, 146.96, 138.10, 136.12, 131.97, 131.95, 131.35, 131.21, 130.16, 128.99, 128.65, 125.74, 125.30, 125.19, 110.74, 110.67, 54.68, 33.78, 26.36, 19.56. ³¹P NMR (121 MHz, Chloroform-d) δ 3.00. MS (ESI) m/z calculated for C₂₆H₂₅N₂O₂P [M+H]⁺ 429.5, found 429.5.

### NR₇₀₇

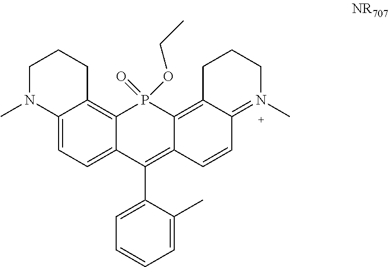

Dark green solid. ¹H NMR (300 MHz, Chloroform-d) δ 7.49-7.30 (m, 3H), 7.05 (dd, J=11.8, 7.3 Hz, 1H), 6.94 (ddd, J=9.4, 6.7, 2.6 Hz, 2H), 6.67 (d, J=9.6 Hz, 2H), 3.81 (dt, J=13.8, 7.1 Hz, 4H), 3.76-3.61 (m, 4H), 3.33 (s, 7H), 2.12 (p, J=5.9 Hz, 4H), 2.03 (s, 3H), 1.30 (t, J=7.0 Hz, 3H). ¹³C NMR (75 MHz, Chloroform-d) δ 163.62, 163.50, 163.34, 163.23, 159.69, 159.17, 158.65, 158.13, 153.80, 153.62, 139.73, 139.64, 139.59, 139.50, 136.54, 136.52, 135.95, 135.78, 133.34, 133.32, 133.26, 133.23, 130.54, 130.42, 129.99, 129.95, 129.43, 129.25, 128.98, 128.40, 128.36, 125.95, 125.91, 124.73, 124.68, 124.62, 124.57, 121.07, 117.26, 114.49, 114.46, 113.44, 109.63, 63.47, 63.32, 63.24, 52.61, 40.96, 25.51, 25.43, 20.55, 19.40, 19.18, 16.21, 16.10. ³¹p NMR (121 MHz, Chloroform-d) δ 18.81. MS (ESI) m/z calculated for C₃₂H₃₄F₃N₂O₄P [M-CF₃COO]⁺485.6, found 485.7.

### NR₇₄₀

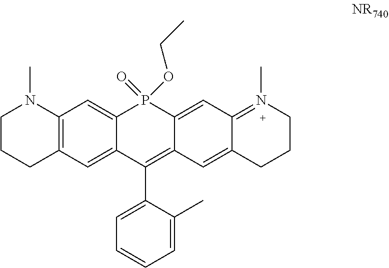

Green solid. ¹H NMR (300 MHz, Methanol-d₄) δ 7.65-7.37 (m, 5H), 7.16 (d, J=7.5 Hz, 1H), 6.75-6.64 (m, 2H), 4.23-4.04 (m, 2H), 3.72 (t, J=5.5 Hz, 4H), 3.45 (s, 6H), 2.69-2.49 (m, 4H), 1.98 (p, J=6.1 Hz, 4H), 1.38-1.23 (m, 3H). ¹³C NMR (75 MHz, Methanol-d₄) δ 162.17, 162.05, 152.96, 152.78, 136.33, 136.19, 135.95, 135.52, 134.71, 134.64, 133.04, 132.97, 130.28, 130.18, 129.33, 128.25, 126.92, 126.90, 126.79, 125.78, 125.57, 124.13, 124.02, 123.87, 123.76, 117.45, 117.36, 62.20, 62.12, 52.51, 39.09, 26.60, 20.17, 18.10, 15.28, 15.19. ³¹P NMR (121 MHz, Methanol-d₄) δ 14.98. MS (ESI) m/z calculated for C₃₂H₃₄F₃N₂O₄P [M-CF₃COO]⁺485.6, found 485.6.

### NR₇₃₅

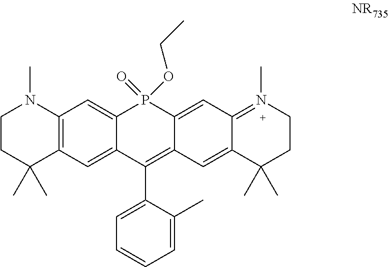

¹H NMR (300 MHz, Chloroform-d) δ 7.56-7.34 (m, 5H), 7.16 (d, J=7.8 Hz, 1H), 6.90 (dd, J=11.0, 6.8 Hz, 2H), 4.15 (h, J=6.9 Hz, 2H), 3.71 (h, J=9.5 Hz, 4H), 3.45 (s, 6H), 2.11 (s, 1H), 2.01 (s, 2H), 1.77 (h, J=8.1 Hz, 4H), 1.34 (dt, J=10.9, 7.0 Hz, 3H), 1.03 (s, 12H). ¹³C NMR (75 MHz, Chloroform-d) δ 163.56, 163.44, 161.04, 160.55, 160.06, 159.56, 151.85, 151.67, 135.75, 135.72, 135.70, 135.64, 135.44, 135.33, 135.26, 134.92, 134.90, 134.86, 134.40, 134.37, 134.26, 134.23, 133.66, 133.59, 130.60, 130.22, 129.76, 129.18, 128.45, 126.05, 125.71, 124.52, 124.42, 124.25, 124.14, 117.90, 117.82, 117.73, 114.06, 62.10, 62.02, 49.28, 40.61, 34.11, 31.63, 31.60, 28.66, 28.58, 28.33, 28.24, 19.40, 19.32, 16.38, 16.28. ³¹P NMR (121 MHz, Chloroform-d) δ 13.08, 13.00. MS (ESI) m/z calculated for C₃₆H₄₂F₃N₂O₄P [M-CF₃COO]⁺541.7, found 541.8.

### NR₇₀₂

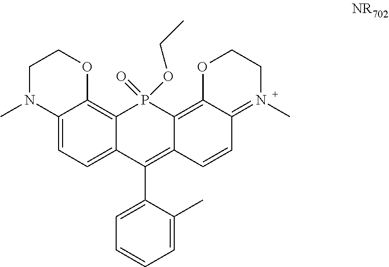

¹H NMR (300 MHz, Chloroform-d) δ 7.45 (t, J=7.4 Hz, 1H), 7.39-7.30 (m, 2H), 7.07 (dd, J=8.2, 2.0 Hz, 1H), 6.86 (ddd, J=10.0, 6.2, 4.0 Hz, 2H), 6.65 (d, J=9.3 Hz, 2H), 4.81-4.55 (m, 7H), 4.53-4.37 (m, 4H), 4.07-3.77 (m, 4H), 3.34 (s, 6H), 2.06 (d, J=3.6 Hz, 3H), 1.42 (t, J=7.1 Hz, 3H). ¹³C NMR (75 MHz, Chloroform-d) δ 148.64, 148.63, 145.93, 145.82, 137.61, 137.47, 136.41, 136.37, 130.35, 130.30, 129.67, 129.48, 129.21, 125.68, 125.61, 125.34, 125.26, 118.67, 116.96, 113.56, 64.22, 64.20, 64.13, 64.11, 63.77, 49.19, 39.55, 19.49, 19.36, 16.29, 16.27, 16.19, 16.18. ³¹P NMR (121 MHz, Chloroform-d) δ 14.34, 14.04. MS (ESI) m/z calculated for C₃₀H₃₀F₃N₂O₆P [M-CF₃COO]⁺489.5, found 489.5.

### General Synthetic Procedure B

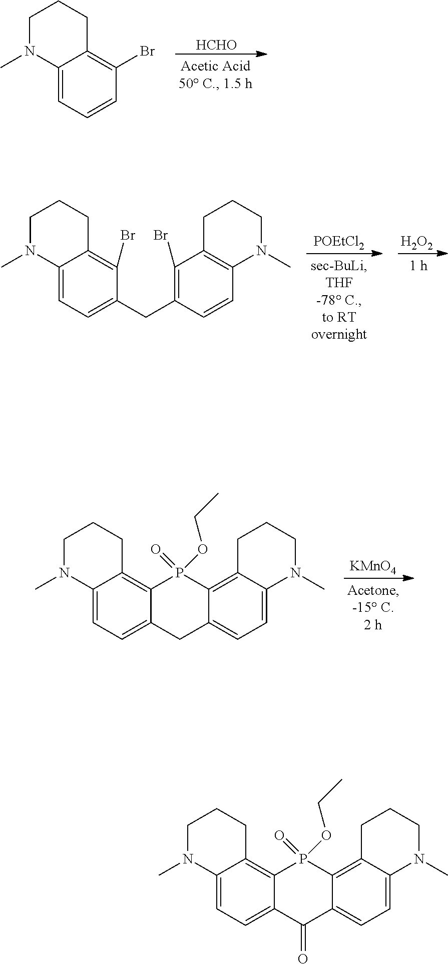

bis(5-bromo-1-methyl-1,2,3,4-tetrahydroquinolin-6-yl)methane

5-bromo-1-methyl-1,2,3,4-tetrahydroquinoline (2.26 g, 10 mmol) was dissolved in acetic acid (30 mL). Formaldehyde solution (37 wt. % in H₂O, 1.5 g, 50 mmol) was added and the resulting mixture was heated at 50° C. for 90 min. The reaction was cooled down to room temperature and the precipitate was filtered and washed with acetic acid and methanol. The resulting white solid (1.55 g, 67%) was then dried under vacuum and was used directly for the next step.

¹H NMR (400 MHz, Chloroform-d) δ 6.73 (d, J=8.5 Hz, 2H), 6.52 (d, J=8.5 Hz, 2H), 4.06 (s, 2H), 3.32-3.12 (m, 4H), 2.89 (d, J=2.4 Hz, 10H), 2.04 (dt, J=12.2, 6.6 Hz, 4H). ¹³C NMR (101 MHz, Chloroform-d) δ 146.82, 128.40, 128.36, 127.73, 122.70, 110.28, 50.90, 42.43, 39.75, 29.52, 22.68. MS (ESI) m/z calculated for C₂₁H₂₄Br₂N₂[M+H]⁺ 465.3, found 465.6.

### 14-ethoxy-4,10-dimethyl-1,3,4,10,11,12,13-heptahydrophosphinino[2,3-f:6,5-f′]diquinolin-7(2H)-one 14-oxide

In a flame dried round bottom flask charged with bis(5-bromo-1-methyl-1,2,3,4-tetrahydroquinolin-6-yl)methane (1.8 g, 3.87 mmol), anhydrous THF (30 mL) was add and the temperature was lowered to −78° C. sec-Butyllithium (1.4 M in cyclohexane, 6.1 mL, 8.52 mmol) was then added dropwise into the reaction over 15 min and the mixture was stirred for 1 h followed by dropwise addition of ethyl dichlorophosphite (490 μL, 4.26 mmol) over 10 min. The reaction was kept at the same temperature for 3 hrs before the dry ice/acetone bath was removed, and the flask was slowly warmed to room temperature overnight. The next day, the flask was cooled to 0° C., hydrogen peroxide (50 wt. % in H₂O, 2.5 mL) was added dropwise into the reaction, and the reaction was stirred for 1 h. Saturated sodium sulfite solution was then carefully added to quench the reaction and ethyl acetate was used for extraction. The organic layer was collected, dried over sodium sulfate, filtered, and the solvent was removed. The resulting solid was further dissolved in acetone and cooled to −15° C. in an ethylene glycol/dry ice bath followed by slow addition of KMnO₄ (1.22 g, 7.74 mmol) over 2 hrs. After the last portion was added, the reaction was stirred for an additional 15 min before DCM was added. The reaction was allowed to warm up to room temperature and the brown mixture was then filtered and washed with DCM. Solvent was removed and flash chromatography (0˜70% DCM/Hexane with 10% Acetone and 0.5% triethylamine) yielded the pure product as a bright yellow solid (428 mg, 27%).

¹H NMR (400 MHz, Chloroform-d) δ 8.24 (dd, J=9.0, 6.5 Hz, 2H), 6.80 (d, J=9.0 Hz, 2H), 3.59 (ddt, J=29.1, 14.6, 7.6 Hz, 4H), 3.43 (tq, J=10.0, 5.3 Hz, 4H), 3.27-3.17 (m, 2H), 3.04 (s, 6H), 2.17-1.93 (m, 4H), 1.15 (t, J=7.0 Hz, 3H). ¹³C NMR (101 MHz, Chloroform-d) δ 180.16, 150.00, 149.87, 129.83, 129.72, 128.76, 127.58, 125.55, 125.47, 124.61, 124.54, 113.63, 113.61, 62.13, 62.07, 51.05, 39.32, 25.67, 25.61, 21.65, 16.16, 16.08. ³¹P NMR (162 MHz, Chloroform-d) δ 20.69. MS (ESI) m/z calculated for C₂₃H₂₇N₂O₃P [M+H]⁺ 411.5, found 411.2.

bis(4-bromo-1-methylindolin-5-yl) methane

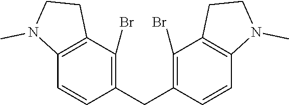

White solid. ¹H NMR (400 MHz, Chloroform-d) δ 6.73 (d, J=7.9 Hz, 2H), 6.32 (d, J=7.9 Hz, 2H), 4.00 (s, 2H), 3.37 (t, J=8.3 Hz, 4H), 3.02 (t, J=8.3 Hz, 4H), 2.75 (s, 6H). ¹³C NMR (101 MHz, Chloroform-d) δ 152.72, 131.35, 129.55, 128.54, 122.09, 105.85, 55.46, 40.12, 36.43, 30.97. MS (ESI) m/z calculated for C₂₁H₂₄Br₂N₂[M+H]⁺ 465.3, found 465.7.

12-ethoxy-3,9-dimethyl-2,3,9,10,11-pentahydrophosphinino[2,3-e:6,5-e′]diindol-6(1H)-one 12-oxide

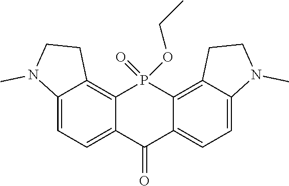

Yellow solid. ¹H NMR (400 MHz, Chloroform-d) δ 8.24 (dd, J=8.6, 5.9 Hz, 2H), 6.57 (dd, J=8.6, 0.9 Hz, 2H), 3.74-3.60 (m, 8H), 2.91 (s, 6H), 1.16 (t, J=7.1 Hz, 3H). ³¹P NMR (162 MHz, Chloroform-d) δ 15.91. MS (ESI) m/z calculated for C₂₃H₂₇N₂O₃P [M+H]⁺ 411.5, found 411.2.

### General Synthetic Procedure C

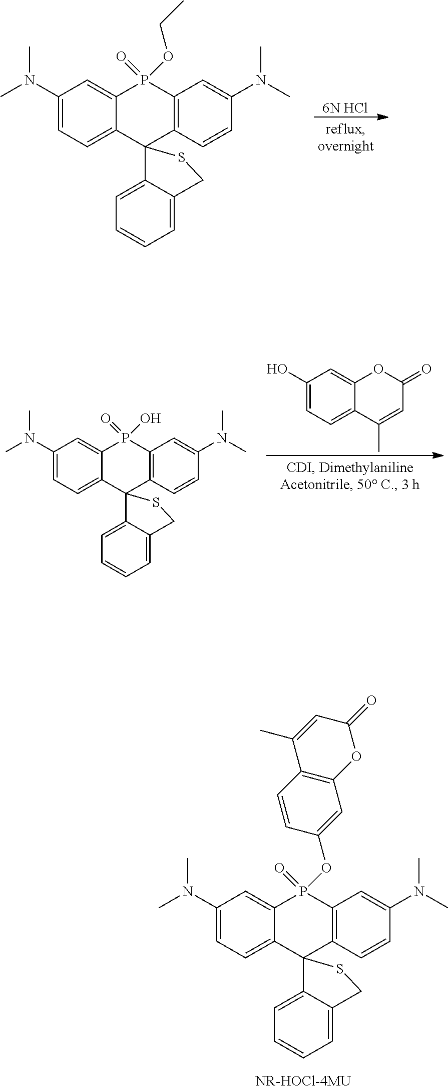

NR—HOCl (52 mg, 0.1 mmol) was dissolved in degassed 6 N HCl and the mixture was refluxed overnight. After removing the solvent, the yellow-green solid was used directly without further purification. 4-Methylumbelliferone (176 mg, 1 mmol) and CDI (162 mg, 1 mmol) along with the crude were dissolved in acetonitrile (1 mL). Dimethylaniline (127 μL, 1 mmol) was added dropwise to the solution and the mixture was heated to 50° C. for 3 h. Solvent was removed, and the mixture separated by HPLC to give 42 mg (yield: 70.6%) light green solid after lyophilization.

¹H NMR (400 MHz, DMSO-d₆) δ 7.75 (d, J=8.8 Hz, 1H), 7.55 (d, J=8.2 Hz, 1H), 7.43 (t, J=7.5 Hz, 1H), 7.31 (t, J=7.2 Hz, 1H), 7.27-7.21 (m, 1H), 7.19-7.09 (m, 3H), 6.87 (dd, J=9.2, 3.1 Hz, 2H), 6.77 (d, J=7.7 Hz, 1H), 6.64 (t, J=8.4 Hz, 2H), 6.37-6.29 (m, 1H), 4.46 (s, 2H), 2.91 (s, 12H), 2.39 (s, 3H). ¹³C NMR (101 MHz, DMSO-d₆) δ 160.14, 159.08, 158.70, 154.11, 153.97, 153.89, 153.55, 148.71, 148.57, 147.40, 142.92, 137.65, 137.56, 130.83, 130.70, 128.44, 128.35, 127.33, 127.31, 126.31, 125.60, 125.04, 117.91, 117.86, 117.78, 117.18, 117.03, 114.31, 113.72, 111.76, 111.68, 109.08, 109.03, 65.80, 65.70, 40.39, 37.67, 18.68. ³¹P NMR (162 MHz, DMSO-d₆) δ 19.05. MS (ESI) m/z calculated for C₃₄H₃₁N₂O₄PS [M+H]⁺ 595.7, found 595.9.

### NR—HOCl-1541B

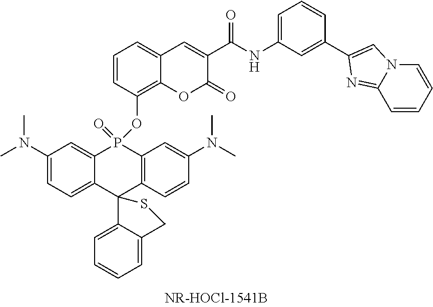 [SMILES: CN(C)c1ccc2c(c1)P(=O)(Oc1cccc3cc(C(=O)Nc4cccc(-c5cn6ccccc6n5)c4)c(=O)oc13)c1cc(N(C)C)ccc1C21SCc2ccccc21]

1541B was obtained through a previously published procedure and coupled to NR—HOCl using General Synthetic Procedure C.

¹H NMR (400 MHz, DMSO-d₆) δ 10.82 (s, 1H), 8.93-8.83 (m, 2H), 8.81 (s, 1H), 8.35 (t, J=2.0 Hz, 1H), 7.87 (ddd, J=32.5, 17.0, 6.9 Hz, 5H), 7.75 (d, J=7.9 Hz, 1H), 7.63 (t, J=7.9 Hz, 1H), 7.57-7.37 (m, 5H), 7.29 (t, J=7.9 Hz, 1H), 7.18 (dd, J=14.9, 2.9 Hz, 2H), 6.94 (dd, J=9.2, 2.9 Hz, 2H), 6.77-6.69 (m, 3H), 4.49 (s, 2H), 2.97 (s, 12H). ¹³C NMR (101 MHz, DMSO-d₆) δ 160.80, 159.77, 158.88, 158.53, 148.93, 148.80, 148.38, 147.59, 142.42, 139.43, 137.80, 137.71, 131.52, 131.40, 130.81, 129.41, 128.44, 128.30, 127.09, 126.79, 126.16, 125.65, 125.55, 124.89, 122.70, 122.08, 121.21, 120.50, 118.14, 117.98, 117.88, 114.97, 113.44, 111.95, 110.97, 65.81, 40.39, 37.94. ³¹P NMR (162 MHz, DMSO-d₆) δ 20.44, 20.42. MS (ESI) m/z calculated for C₄₇H₃₈N₅O₅PS [M+H]⁺ 816.9, found 817.1.

### Synthesis of 6-NHS—NR₆₆₆

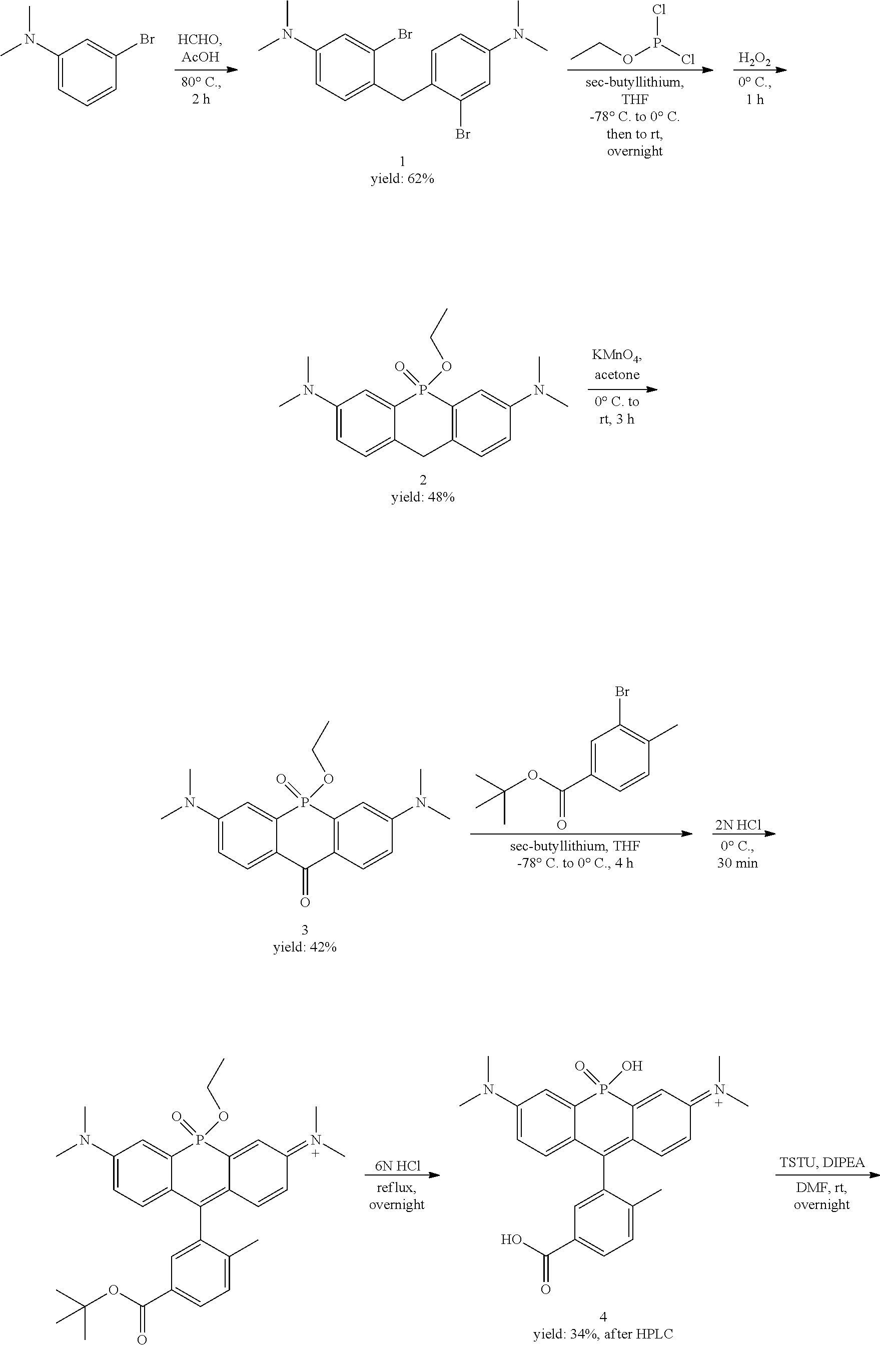

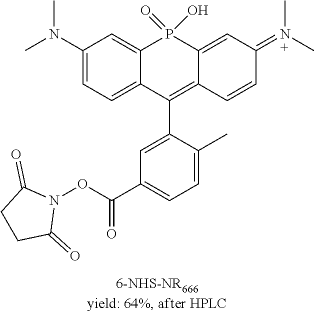

### 4,4′-methylenebis(3-bromo-N,N-dimethylaniline) (1)

3-bromo-N,N-dimethylaniline (10 g, 50 mmol) and formaldehyde (37 wt. % in H₂O, 7.5 g, 250 mmol) were dissolved in acetic acid (200 mL) and the mixture was stirred at 80° C. for 1 h. Solvent was removed by evaporation and saturated sodium bicarbonate was added. The resulting mixture was extracted by DCM and the organic layer was collected, dried over sodium sulfate, and filtered. Solvent was removed and flash chromatography (10% DCM in hexane) was performed to yield the product as a white solid (6.39 g, yield: 62%).

### 3,7-bis(dimethylamino)-5-ethoxy-10H-acridophosphine 5-oxide (2)

To a flame dried round bottom flask charged with 4,4′-methylenebis(3-bromo-N,N-dimethylaniline) (1) (5 g, 12.13 mmol), anhydrous THF (80 mL) was add and the temperature was lowered to −78° C. with a dry ice/acetone bath. Sec-butyllithium (1.4 M in cyclohexane, 19.1 mL, 26.69 mmol) was then added dropwise into the reaction over 20 min and the mixture was stirred for an additional 1 h followed by dropwise addition of ethyl dichlorophosphite (1.525 mL, 13.34 mmol) over 40 min. The reaction was allowed to proceed for 3 h and then the dry ice/acetone bath was replaced by an ice bath and allowed to proceed overnight. Hydrogen peroxide (50 wt. % in H₂O, 8 mL) was added dropwise into the reaction and the reaction was stirred at 0° C. for 1 h. Saturated sodium sulfite solution was then added to quench the reaction and ethyl acetate was used for extraction. The organic layer was collected, dried over sodium sulfate, filtered, and the solvent was removed. The resulting mixture was purified by the flash chromatography (ethyl acetate as eluent) to afford a colorless oil (2 g, yield: 48%).

### 3,7-bis(dimethylamino)-5-ethoxy-10H-acridophosphin-10-one 5-oxide (3)

3,7-bis(dimethylamino)-5-ethoxy-10H-acridophosphine 5-oxide (2) (1.03 g, 3.01 mmol) was further dissolved in acetone and cooled to 0° C. followed by portion-wise addition of KMnO₄ (1.43 g, 9.03 mmol) over 2 h. After the last portion was added, the reaction was allowed to warm to room temperature and stirred for another 1 h. The mixture was then filtered and washed with DCM. Solvent was removed and flash chromatography (5-10% acetone in DCM with 0.5% trimethylamine) yielded the pure product as a yellow solid (453 mg, yield: 42%).

### 6-carboxyl-NR₆₆₆ (4)

tert-Butyl-3-bromo-4-methylbenzoate (1.356 g, 5 mmol) was dissolved in anhydrous THF (40 mL) and the temperature was lowered to −78° C. with an acetone/dry ice bath. After stirring for 10 min, sec-butyl lithium (1.4 M in cyclohexane, 3.57 mL, 5 mmol) was added dropwise within 10 min and the mixture was stirred at the same temperature for 1 h. 3,7-bis(dimethylamino)-5-ethoxy-10H-acridophosphin-10-one 5-oxide (3) (358 mg, 1 mmol) was dissolved in 40 mL THF using sonication and added dropwise into the reaction mixture. The temperature was then allowed to rise to 0° C. and the reaction was stirred for an additional 4 h on ice. 2 N HCl (15 mL) was used to quench the reaction and the pH was adjusted to 3-4 by addition of a saturated sodium bicarbonate solution. The mixture was extracted with DCM three times. The DCM layer was collected, dried over sodium sulfate, filtered, and solvent was evaporated. The resulting green solid was dissolved in 6 N HCl (10 mL) and refluxed overnight. After removing the solvent, the mixture was dissolved in HPLC solvent (50/50 water:acetonitrile with 0.1% trifluoroacetic acid) and purified by HPLC. A dark blue solid (153 mg, yield: 34%) was obtained after lyophilization.

### 6-NHS—NR₆₆₆ (5)

6-carboxyl-NR₆₆₆ solid (135 mg, 0.3 mmol) was dissolved in DMF (2 mL), DIPEA (435 μL, 2.5 mmol) and TSTU (301 mg, 1 mmol) were added and the reaction was stirred at room temperature for 6 h. After removing the solvent, the mixture was dissolved in HPLC solvent (50/50 water:acetonitrile with 0.1% trifluoroacetic acid) and purified by HPLC. After lyophilization, a dark blue NHS-ester solid (105 mg, yield: 64%) was obtained.

### Synthesis of NR₇₅₁

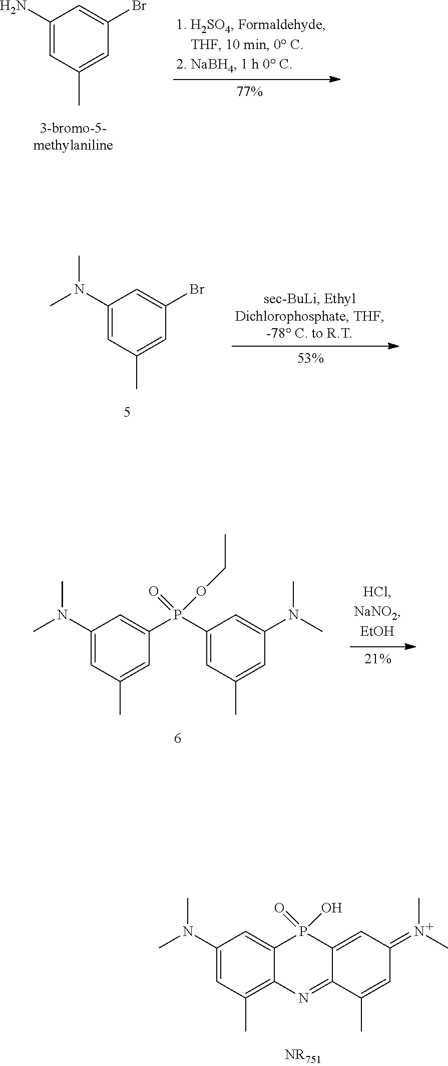

### 3-bromo-5-methyl-N,N-dimethylaniline (5)

Aqueous H₂SO₄ (3M, 16 mL) was poured into a stirred solution of aqueous formaldehyde (37%, 6 mL, 80.7 mmol) in THF (100 mL), and the mixture was immersed in an ice bath and stirred for 10 min. A THF solution of 3-bromo-5-methylaniline (5 g, 26.9 mmol) was added dropwise to the mixture, and the resultant mixture was stirred at 0° C. for an additional 10 min. Sodium borohydride (4.1 g, 107.6 mmol) was added portion wise while maintaining the reaction mixture at 0° C. The resulting mixture was warmed up to room temperature and reacted for 1 h. 1M NaOH (20 mL) was added to the mixture to quench the reaction. The resulting mixture was extracted with Ethyl acetate (100 mL×2). The combined organic phase was washed with water and brine, then dried over anhydrous Na₂SO₄ and filtered. The solvent was evaporated in vacuo. The resulting residue was further purified by column chromatography eluting with hexane/ethyl acetate=100:1. Yield 4.4 g (77%) of colorless liquid.

¹H NMR (400 MHz, CDCl₃): δ (ppm) 6.72-6.74 (m, 2H), 6.49 (s, 1H), 2.97 (s, 6H), 2.34 (s, 3H). ¹³C NMR (100 MHz, CDC₃): 151.72, 140.49, 123.30, 120.19, 112.57, 111.91, 40.56, 21.80.

### Ethyl bis(3-(dimethylamino)-5-methylphenyl)phosphinate (6)

A solution of 3-bromo-5-methyl-N,N-dimethylaniline 5 (500 mg, 2.3 mmol) in anhydrous THF (5 mL) was cooled to −78° C. under nitrogen atmosphere, then sec-BuLi (1.7 mL, 1.4 M) was injected dropwise over 10 min. The mixture was stirred for 1 h at −78° C., then ethyl dichlorophospate (150 μL, 1.2 mmol) was injected into the mixture. Subsequently, the mixture was stirred at −78° C. for 6 h then reacted for an additional 12 h at room temperature. After that, the reaction was quenched with sat. aqueous NH₄Cl. The resulting mixture was extracted with Ethyl acetate (20 mL×2), and the combined organic phase was washed with brine then dried over anhydrous Na₂SO₄. The solvent was evaporated through the vacuum, and the resulting residue was further purified through column chromatography eluting with Ethyl acetate to yield a yellowish oil, 444 mg (53%).

¹H NMR (600 MHz, CDC₃) δ 7.01 (d, J=14.3 Hz, 1H), 6.92 (d, J=12.0 Hz, 1H), 6.62 (s, 1H), 4.07 (p, J=7.2 Hz, 1H), 2.93 (s, 6H), 2.30 (s, 3H), 1.35 (t, J=7.0 Hz, 2H). ¹³C NMR (151 MHz, CDC₃) δ 187.80, 150.38, 150.28, 138.89, 138.79, 132.57, 131.68, 120.18, 120.11, 116.47, 112.67, 112.60, 110.03, 60.81, 40.52, 21.80, 16.54. ³¹P NMR (201 MHz, CDC₃) δ 32.64. (85% H₃PO₄ in CDC₃ as external standard).

### NR-Oxazine₇₅₁

Compound 5 (400 mg, 1.1 mmol) was dissolved in anhydrous ethanol (8 mL). To this solution, an aqueous sodium nitrite solution (92 mg, 1.3 mmol in 2.6 mL water) was added at 0° C. and stirred at 0° C. for 10 minutes. Then concentrated hydrochloric acid (380 mL) was added to the solution at 0° C. dropwise. After reacting at 0° C. for 10 minutes, warmed up the reaction mixture to 50° C. and reacted for 12 hours. After completion of the reaction, the reaction mixture was poured into 10 mL water and adjusted the pH to 7 by aqueous 1M sodium hydroxide solution. The resulting aqueous solution was washed with ethyl acetate three times followed by lyophilization. The resulting solid was dissolved with 50% methanol in water and purified with HPLC eluted from 50% water (containing 0.1% TFA) in methanol to 100% methanol with monitoring absorbance at 700 nm. The resulting solution was rotary vaporized and lyophilized to furnish dark green solid, 79 mg (21%). ¹H NMR (600 MHz, CD₃OD) δ 7.46 (d, J=13.5 Hz, 2H), 7.01 (s, 1H), 6.62 (s, 1H), 3.48 (s, 12H), 2.55 (s, 6H), 2.30 (s, 3H), 1.30 (s, 1H). ¹³C NMR (151 MHz, CD₃OD) δ 155.98, 155.89, 152.00, 151.94, 141.26, 141.22, 116.32, 115.69, 40.39, 17.97. ³¹P NMR (201 MHz, CDC₃) δ 2.81. (85% H₃PO₄ in CDC₃ as external standard).

### Example 2: Phosphinate-Containing Thiazine-Based Dyes Bearing Azetidine Auxochromes

### Compound Synthesis and Characterization

### Synthesis of Az-NR₇₅₁

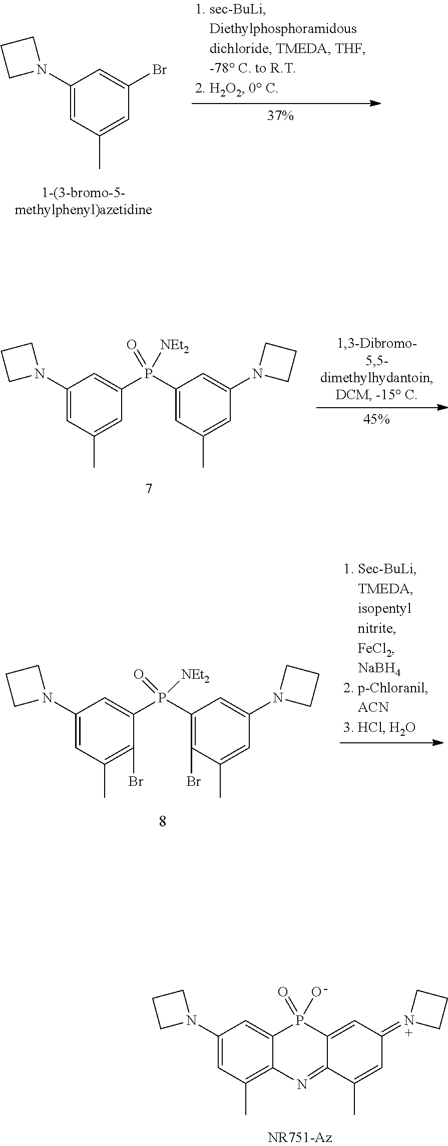

### P,P-bis(3-(azetidin-1-yl)-5-methylphenyl)-N,N-diethylphosphinic amide (7)

In a fume dried flask, to a solution of 1-(3-bromo-5-methylphyl)azetidine (1.3 g, 5.8 mmol) and tetramethylethylenediamine (3.9 mL, 26.1 mmol) in anhydrous THF, sec-BuLi (1.4 M in cyclohexane) was injected dropwise at −78° C. and reacted at that temperature for 3 hours. Then diethylphosphoramidous dichloride (337 μL, 3.5 mmol) was added to reaction mixture at −78° C. and stirred for another 6 hours. After that, the reaction mixture was gradually warmed up to room temperature and stirred for extra 12 hours. The reaction was quenched by adding 1 mL of water dropwise. The resulting solution was cooled to 0° C., and 1.5 mL hydrogen peroxide (50 wt % in water) was added dropwise. The reaction mixture was stirred at 0° C. for 1 h and quenched by saturated aqueous sodium sulfite solution. The resulting suspension was filtered, the filtrate was diluted by ethyl acetate and washed with brine. The organic phase was dried through anhydrous sodium sulfate. Followed by evaporating the solvent in vacuum, the residue was further purified by silica gel chromatography eluted with 20% hexane in ethyl acetate. 0.53 g (37%) of light-yellow foam was furnished. ¹H NMR (500 MHz, CD₃OD) δ 6.92 (d, J=12.0 Hz, 2H), 6.67 (d, J=13.2 Hz, 2H), 6.45 (d, J=7.8 Hz, 2H), 3.88-3.82 (m, 8H), 3.06 (dq, J=10.9, 7.1 Hz, 4H), 2.39-2.33 (m, 4H), 2.31-2.28 (m, 6H), 1.11 (t, J=7.0 Hz, 6H). ¹³C NMR (151 MHz, CD₃OD) δ 153.98, 153.88, 140.61, 140.51, 133.42, 132.56, 122.78, 122.72, 116.79, 116.78, 113.33, 113.26, 53.60, 40.71, 40.68, 21.78, 17.98, 14.65, 14.62. ³¹P NMR (201 MHz, CD₃OD) δ 36.83 (neat 85% H₃PO₄ internal standard). HR-ESIMS m/z calculated for C₂₄H₃₅N₃OP⁺[M+H⁺]: 412.2512, found: 412.2608.

### P,P-bis(5-(azetidin-1-yl)-2-bromo-3-methylphenyl)-N,N-diethylphosphinic amide (8)

To a solution of P,P-bis(3-(azetidin-1-yl)-5-methylphenyl)-N,N-diethylphosphinic amide 1 (531 mg, 1.3 mmol) in anhydrous DCM (20 mL), 1,3-dibromo-5,5-dimethylhydantoin (369 mg, 1.3 mmol) was added portionwise at −15° C. The mixture was stirred at that temperature for 2 h then quenched with sat. NaHCO₃ solution. Subsequently, the mixture was extracted with DCM followed by washing with brine. After that, the organic phase was further dried over anhydrous Na₂SO₄. The solvent was evaporated through the vacuum, and the resulting residue was further purified through column chromatography eluting with Ethyl acetate to yield light yellow foam, 330 mg (45%). ¹H NMR (600 MHz, CD₃OD) δ 6.59 (d, J=2.8 Hz, 2H), 6.51 (dd, J=15.7, 2.8 Hz, 2H), 3.83 (t, J=7.3 Hz, 8H), 3.21 (dq, J=11.1, 7.0 Hz, 2H), 2.40-2.35 (m, 10H), 1.13 (t, J=7.1 Hz, 6H). ¹³C NMR (151 MHz, CD₃OD) δ 152.19, 152.09, 141.46, 141.38, 133.21, 132.24, 119.00, 118.94, 114.73, 114.69, 63.09, 63.05, 53.78, 23.96, 23.95, 17.84, 16.81, 16.77. ³¹P NMR (201 MHz, CD₃OD) δ 31.26. (neat 85% H₃PO₄ internal standard) HR-ESIMS m/z calculated for C24H33Br2N3OP⁺[M+H⁺]: 568.0723, found: 568.0719.

### Az-NR₇₅₁

Compound 8 (280 mg, 0.5 mmol) was dissolved in anhydrous THF (20 mL). To this solution, tetramethylethylenediamine (552 μL, 3.7 mmol) was added. Sequentially, sec-BuLi (882 μL, 1.2 mmol) was added to the solution dropwise at −78° C. After reacting at −78° C. for 3 hours, isopentyl nitrite (100 μL, 740.7 μmol) was added and the reaction was stirred at −78° C. for 6 hours then gradually warm to room temperature for another 12 hours. After that, ethanol (10 mL) was added into reaction mixture, Iron (II) chloride (156 mg, 1.2 mmol) and sodium borohydride (28 mg, 740.7 μmol) was added sequentially at 0° C. and the reaction was stirred at that temperature for 2 hours. After completion of the reaction, the solvent was evaporated. To the resulting residue diethyl ether (10 mL) was added. After filtration, the filtrate was concentrated by rotary evaporation, The crude product (133 mg) was used for next step without further purification.

The crude product (39 mg) was dissolved in acetonitrile (10 mL), p-chloranil (34 mg, 137.9 μmol) was added into the reaction and stirred at room temperature for 15 minutes. Then the reaction mixture was added into 0.05 M HCl at 0° C. The reaction was stirred at 0° C. for 1 hour. The resulting suspension was subjected to filtration, and the pH of filtrate was adjusted to 4 and subjected to HPLC purification eluted from 50% water (containing 0.1% TFA) in methanol (containing 0.2% TFA) to 100% methanol (containing 0.2% TFA) with monitoring absorbance at 700 nm. The resulting solution was rotary vaporized and lyophilized to furnish dark green solid, 11 mg (33%). HR-ESIMS m/z calculated for C₂₀H₂₃N₃O₃P⁺[M⁺]: 368.1522, found: 368.1524.

### Properties of the Phosphinate-Containing Thiazine-Based Dyes

To demonstrate the generality of the phosphinate substitution, herein are reported the synthesis and characterization of a phosphinate-containing thiazine-based fluorophore (FIG. 2.1). This dye displays an ˜88 nm red-shift in absorption and emission compared to the parent thiazine (containing sulfur in place of the phosphinate) and demonstrates the generalizability of phosphinates for red-shifting the absorption and emission of dyes. Structural modifications to this scaffold should produce dyes with emission ranging from ˜700-800 nm.

To demonstrate the advantage of phosphinate substitution in the thiazine dye family, it has been investigated whether phosphinate substitution could suppress aggregation of NR₇₅₁ versus the parent thiazine, methylene blue (MB, FIG. 2.2A). A clear deviation of absorbance from linearity in aqueous solutions containing increasing concentrations of MB is observed, while a clear linear increase in absorbance versus concentration of linearity NR₇₅₁ is observed (FIGS. 2.2B-2.2C). Moreover, an increase in H-aggregate formation (as evidenced by a relative increase in a blue-shifted absorbance shoulder) was observed for MB but not NR₇₅₁ (FIG. 2.2D). The H-aggregate peak for MB could be reduce by addition of a surfactant while surfactant did not influence the absorbance of NR₇₅₁ (FIG. 2.2E). Taken together, these data clearly demonstrate the ability to significantly reduce aggregation of thiazine-based dyes via phosphinate substitution.

We further investigated the influence of solvent on NR₇₅₁ absorbance and emission. Interestingly, a trend towards longer wavelength absorption and emission in increasingly polar solvents was observed (FIGS. 2.3A-2.3B). These data demonstrate that changes in NR₇₅₁ absorbance and/or emission could be used to readout the changes in its local environment.

As an initial effort to increase the fluorescence brightness of NR₇₅₁, we synthesized an azetidine-containing derivative based on previous observations that this substituent is capable of rotation within fluorophore scaffold leading increases in quantum yields (FIG. 2.4). Indeed, the resulting derivative Az-NR₇₅₁ displayed a 60% increase in quantum yield relative to NR₇₅₁ (FIG. 2.4). The resulting 60% increase in brightness could enable deep tissue imaging application in living organisms.

### Example 3: Tuning Phosphinate Ester Stability

Replacing the phosphinate ethyl ester in NR₇₀₀ with a thiophosphinate ethyl ester (SNR₇₀₀) results in a 10-fold decrease in the relative rate of phosphinate ester hydrolysis (see FIG. 1.1 for structures and properties of dyes). Thus, thiophosphinate dyes provide longer lived species for imaging applications (e.g., see section of turn-photoacoustic probes).

### Compound Synthesis and Characterization

### Synthesis of SNR₇₀₀

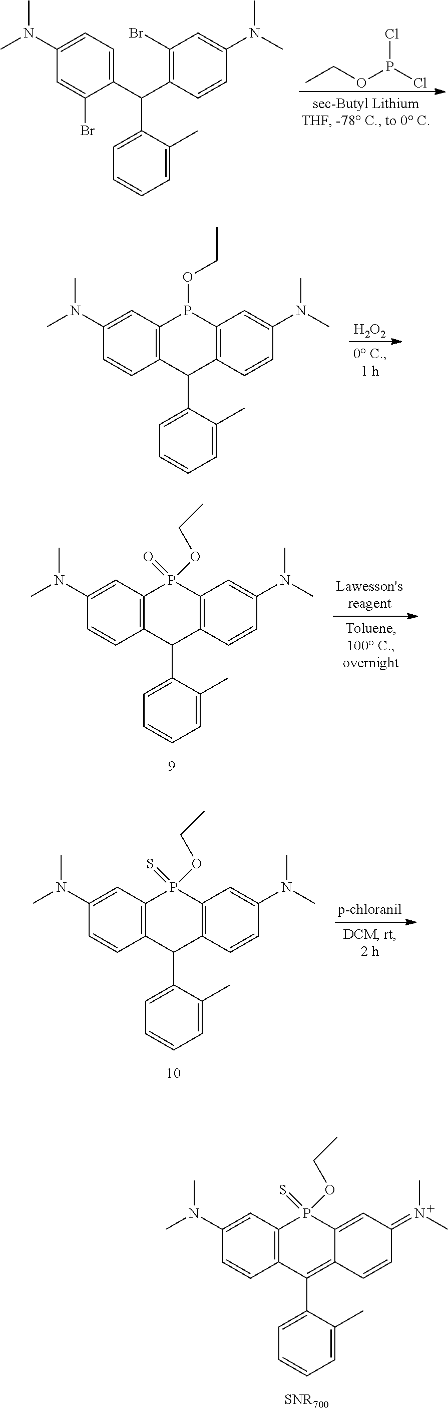

4,4′-(o-tolylmethylene)bis(3-bromo-N,N-dimethylaniline (500 mg, 0.995 mmol, 1 eq.) was dissolved in anhydrous THF (10 mL) and the solution was cooled to −78° C. in an acetone/dry ice bath. After stirring for 10 min, sec-butyllithium (1.4 M in cyclohexane, 1.57 mL, 2.189 mmol, 2.2 eq.) was added dropwise over 10 min and the resulting mixture was further stirred for 1 hr followed by dropwise addition of ethyl dichlorophosphite (0.137 mL, 1,195 mmol, 1.2 eq.) over 10 min. The reaction was allowed to proceed overnight from −78° C. to room temperature. Reaction was cooled to 0° C., and H₂O₂ (1.3 mL, 30%) was added, and stirred for 1 hr. The reaction was then quenched by saturated sodium sulfite solution and extracted using ethyl acetate. The organic layer was collected, dried over sodium sulfate. After removing the solvents under reduced pressure, the resulting mixture was subjected to silica gel column chromatography (0-15% MeOH in CH₂Cl₂) to afford a light green solid (250 mg, 0.58 mmol), which still contained inseparable impurity, yet used in the next transformation without further purification. Intermediate 9 was dissolved in 10 mL anhydrous toluene, and degassed. Lawesson's reagent (139 mg, 0.345 mmol, 0.6 eq.) dissolved in 5 mL anhydrous toluene was added to reaction and stirred at 100° C. overnight. Solvent was removed under reduced pressure and the resulting mixture was flushed through a short silica gel column (0-10% MeOH in CH₂Cl₂) to give 10, which was dissolved in DCM (10 mL). P-chloranil (0.425 g, 1.728 mmol, 3 eq.) was added and the reaction was stirred at room temperature for 2 hr. The resulting mixture was flushed through a short silica gel column (0-15% MeOH in CH₂Cl₂ with 1% CH₃COOH), after which the collections were evaporated to give a dark green solid. The resulting dark green solid was added to HPLC buffer (50% acetonitrile in water with 0.1% trifluoroacetic acid) and clarified by centrifugation. The product peak from HPLC was lyophilized to yield SNR₇₀₀ as a dark green solid (15 mg, 3.35%). A racemic mixture was obtained using this method and it was used for further studies with no further purification.

### Example 4: Phosphinate Dyes with Improved Chemical Stability

### Compound Synthesis and Characterization

### NR Analogs Bearing Bulky Substituents Proximal to the C9 Position

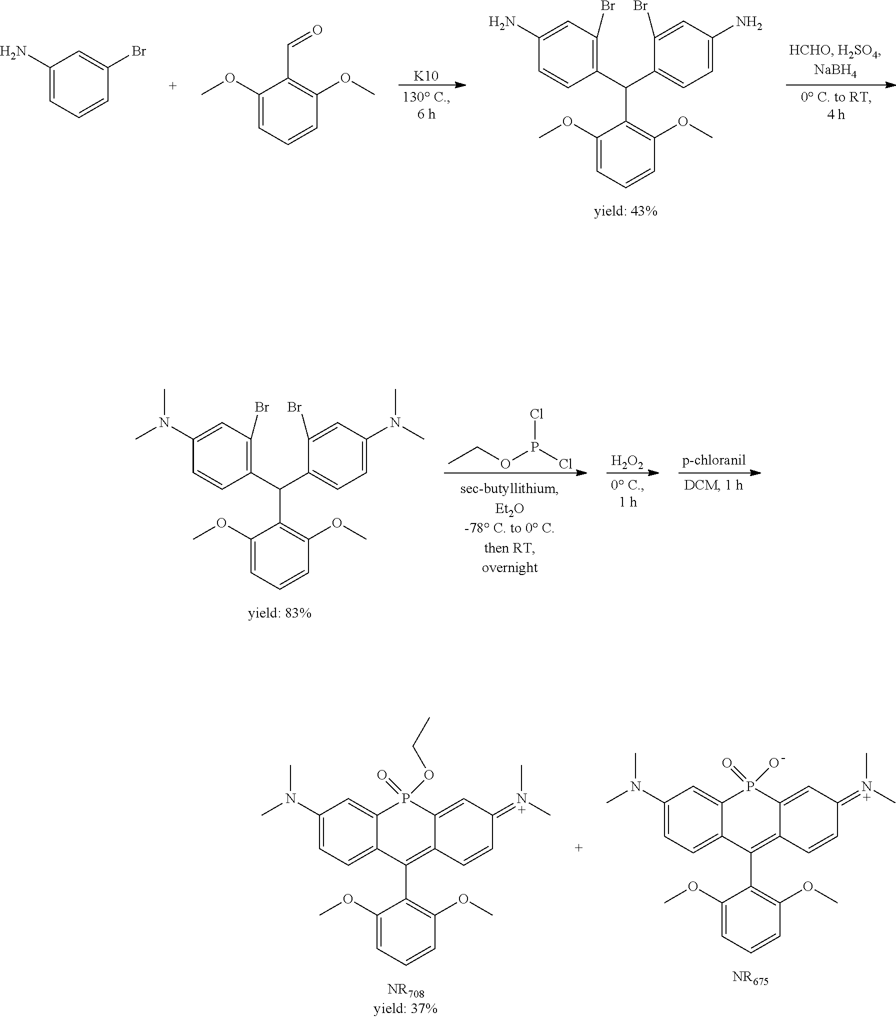

### 4,4′-((2,6-dimethoxyphenyl)methylene)bis(3-bromoaniline)

3-bromoaniline (6.88 g, 40 mmol) and 2,6-dimethoxybenzaldehyde (1.66 g, 10 mmol) were mixed well with montmorillonite K10 (8.5 g) in a 50 mL round bottom flask, the reaction was stirred at 130° C. with nitrogen flow. At the first 1 h, a spatula was frequently used to help the stir bar work in the sticky mixture. Once the mixture became in sand state, and stirred well, the reaction vessel was held at 130° C. for another 5 h. The reaction was cooled to room temperature, diluted with DCM, the mixture was filtered with Celite, and the filter cake was washed with DCM and MeOH. After removing the solvent, the residue was purified with silica gel chromatography (20-50%, ethyl acetate/hexane) afforded white solid (2.12 g, 43%).

¹H-NMR (600 MHz, Chloroform-d) δ 7.20 (t, J=8.3 Hz, 1H), 6.86 (d, J=2.5 Hz, 2H), 6.82 (d, J=8.4 Hz, 2H), 6.55 (d, J=8.3 Hz, 2H), 6.49 (dd, J=8.4, 2.4 Hz, 2H), 6.18 (s, 1H), 3.56 (s, 10H). ¹³C-NMR (151 MHz, Chloroform-d) δ 158.95, 145.26, 132.32, 132.13, 128.06, 125.24, 119.73, 118.77, 113.66, 56.09, 45.64.

### 4,4′-((2,6-dimethoxyphenyl)methylene)bis(3-bromo-N,N-dimethylaniline)

3 M H₂SO₄ (2.5 mL) was added to a stirred HCHO (37%, 890 μL, 12 mmol) in 3 mL THF, mixture was cooled to 0° C. for 10 min. 4,4′-((2,6-dimethoxyphenyl)methylene)bis(3-bromoaniline) (985 mg, 2 mmol) was dissolved in 7.5 mL THF, and added to the stirring mixture dropwise, after which the reaction was left at 0° C. for another 10 min. NaBH₄ (912 mg, 24 mmol) was added portion-wise at 0° C., then the reaction temperature was increased to room temperature. After 1 h reaction, saturated NaHCO₃ was used to adjust the pH to neutral, and the product extracted with DCM (100 mL×2), organic layer was dried over anhydrous Na₂SO₄, and the solvent was removed for column chromatography. White solid (909 mg, 83%) was afforded using EtOAc/Hexane (1:8 to 1:5).

¹H-NMR (600 MHz, Chloroform-d) δ 7.20 (t, J=8.3 Hz, 1H), 6.94-6.89 (m, 4H), 6.57 (dd, J=8.5, 2.1 Hz, 4H), 6.25 (s, 1H), 3.58 (s, 6H), 2.90 (s, 12H). ¹³C-NMR (151 MHz, Chloroform-d) δ 159.11, 149.71, 131.93, 130.42, 128.04, 125.75, 120.28, 116.22, 111.24, 56.30, 45.58, 40.65.

### NR₇₀₈

This compound was a blue solid, synthesized following General Synthetic Procedure A.

¹H-NMR (600 MHz, Methanol-d₄) δ 7.71-7.64 (m, 1H), 7.58 (s, 1H), 7.54-7.46 (m, 1H), 7.32 (dd, J=9.9, 4.9 Hz, 1H), 7.16 (dd, J=8.9, 5.6 Hz, 1H), 6.97 (dd, J=9.9, 2.1 Hz, 1H), 6.78 (dd, J=9.0, 2.2 Hz, 1H), 3.67 (s, 6H), 3.57 (s, 6H), 4.09 (dp, J=9.6, 7.2 Hz, 2H), 1.24 (t, J=7.0 Hz, 3H)¹³C-NMR (151 MHz, Methanol-d₄) δ 164.81, 164.68, 162.81, 162.74, 160.88, 158.43, 158.30, 157.67, 142.35, 142.27, 137.63, 137.53, 131.45, 131.09, 128.56, 127.56, 127.48, 126.90, 126.84, 118.23, 118.17, 117.94, 117.25, 117.22, 115.85, 113.64, 103.79, 103.72, 103.30, 55.10, 40.98, 18.65, 15.77. ³¹P-NMR (201 MHz, Methanol-d₄) δ 12.56, 12.48.

### Chemical Stability of the Dyes

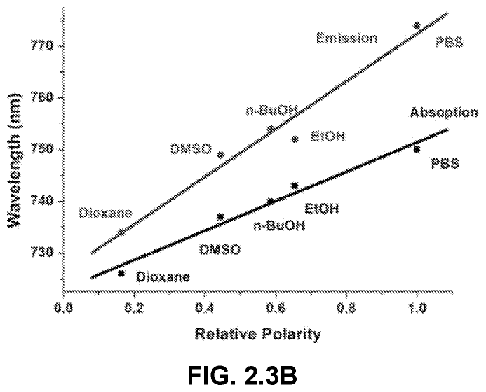

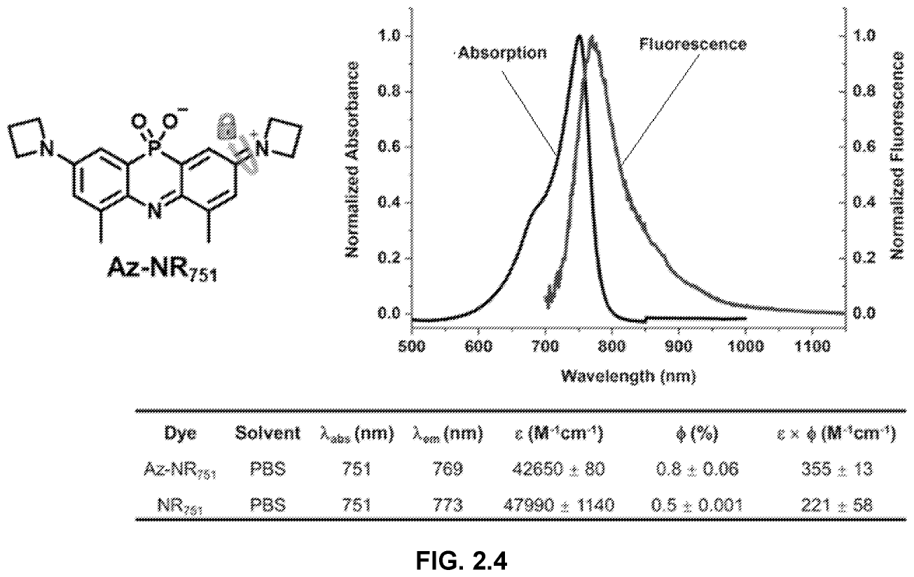

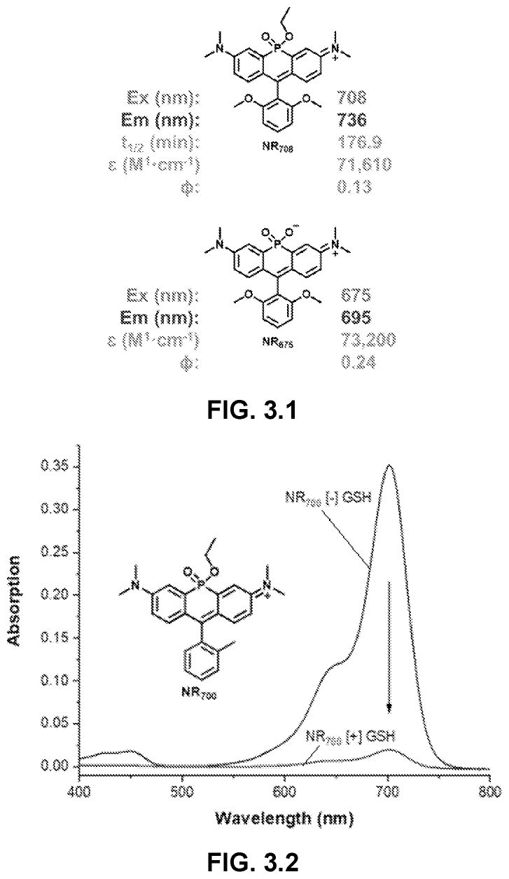

In order to improve the chemical stability of NR dyes to nucleophiles found in biological systems, such as glutathione (GSH), bulky substituents were introduced to protect the C9 position of the dyes from nucleophilic attack. Accordingly, derivatives of NR₇₀₀ and NR₆₆₆, termed NR₇₀₈ and NR₆₇₅, respectively, were synthesized that bear bulky substituents on the pendant phenyl ring proximal to the C9 position of the dye (FIG. 3.1). These analogs display virtually no quenching due to GSH compared to the parent dyes (FIGS. 3.2-3.3). The improved chemical stability of these dyes will provide brighter probes for imaging applications.

### Example 5: Phosphinate Dyes with Increased Quantum Yields

### NR Analogs Bearing Amino Auxochromes

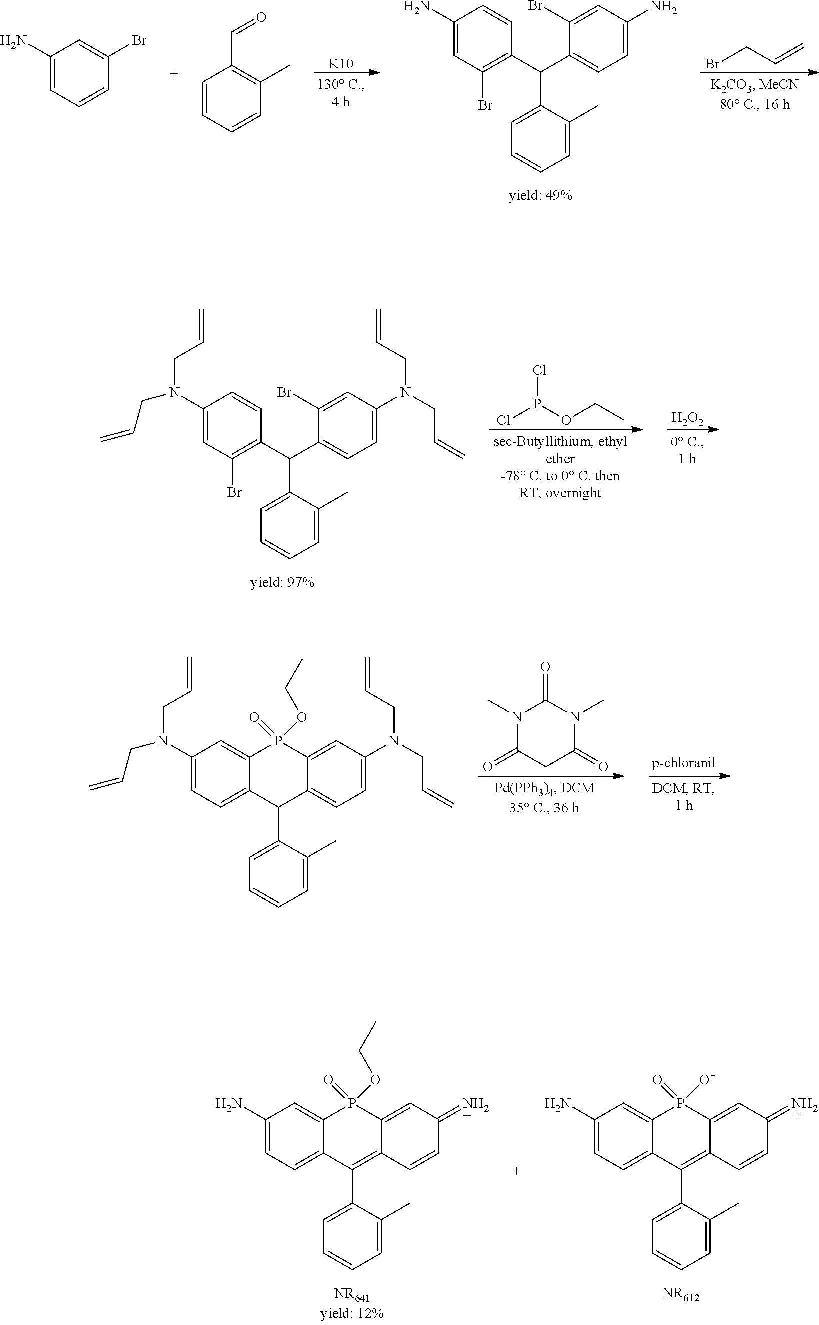

### 4,4′-(o-tolylmethylene)bis(3-bromoaniline)

3-bromoaniline (5.16 g, 30 mmol) and 2-methylbenzaldehyde (1.2 g, 10 mmol) were mixed well with montmorillonite K10 (6.5 g) in a 50 mL round bottom flask, the reaction was stirred at 130° C. with nitrogen flow. At the first 1 h, the spatula was frequently used to help the stir bar work in the sticky mixture. Once the mixture became in sand state, and stirred well, left the reaction at 130° C. for another 3 h. The reaction was cooled to room temperature, diluted with DCM, and the mixture was filtered with Celite, filter cake was washed with DCM and MeOH. After removing the solvent, the residue was purified with silica gel chromatography (0-30%, ethyl acetate/hexane) afforded white solid (2.19 g, 49%).

¹H-NMR (600 MHz, CDC₃) δ 7.17-7.13 (m, 2H), 7.07 (ddd, J=7.2, 6.4, 2.2 Hz, 1H), 6.94 (d, J=2.4 Hz, 2H), 6.71 (dd, J=7.5, 1.2 Hz, 1H), 6.55 (d, J=8.3 Hz, 2H), 6.50 (dd, J=8.3, 2.4 Hz, 2H), 5.92 (s, 1H), 3.61 (s, 4H), 2.19 (s, 3H). ¹³C-NMR (151 MHz, CDC₃) δ 145.97, 141.26, 137.14, 131.85, 131.40, 130.44, 128.80, 126.51, 126.30, 125.76, 119.46, 114.12, 51.64, 19.62.

### 4,4′-(o-tolylmethylene)bis(N,N-diallyl-3-bromoaniline

4,4′-(o-tolylmethylene)bis(3-bromoaniline) (2 g, 4.48 mmol) and K₂CO₃ (2.48 g, 17.92 mmol) were dissolved in 25 mL anhydrous acetonitrile. Allyl bromide (2.72 mL, 31.4 mmol) was added to the mixture dropwise, and the reaction was kept at 70° C. for 14 h. Cooled the reaction to room temperature, K₂CO₃ was filtered, and the solvent was removed. The resulting mixture was purified by silica gel chromatography (1:30, ethyl acetate/hexane) to afforded light yellow oil (2.63 g, 97%). ¹H-NMR (600 MHz, CDC₃) δ 7.17-7.12 (m, 2H), 7.09 (td, J=7.4, 1.9 Hz, 1H), 6.92 (d, J=2.7 Hz, 2H), 6.75 (dd, J=7.5, 1.3 Hz, 1H), 6.61 (d, J=8.7 Hz, 2H), 6.52 (dd, J=8.7, 2.7 Hz, 2H), 5.95 (s, 1H), 5.84 (ddt, J=17.0, 10.2, 4.9 Hz, 4H), 5.21-5.18 (m, 4H), 5.17 (t, J=1.7 Hz, 4H), 3.88 (dt, J=5.0, 1.7 Hz, 8H), 2.23 (s, 3H). ¹³C-NMR (151 MHz, CDC₃) δ 148.32, 141.82, 137.09, 133.72, 131.11, 130.32, 129.43, 128.84, 126.53, 126.28, 125.66, 116.48, 116.37, 111.20, 52.80, 51.40, 19.74.

### NR₆₄₁

In a flame dry 100 mL round bottom flask, 4,4′-(o-tolylmethylene)bis(N,N-diallyl-3-bromoaniline) (2.54 g, 4.2 mmol) was dissolved with 25 mL anhydrous ethyl ether and 25 mL anhydrous THF under the N₂ protection. Cooled the mixture to −78° C. for 10 min, 6.6 mL sec-butyllithium (1.4 M in cyclohexane, 9.24 mmol) was added dropwise within 10 min. The reaction was further kept at −78° C. for 1 h, and ethyl dichlorophosphite (528 μL, 4.62 mmol) was added dropwise. After reacting at −78° C. for two more hours, the reaction was slowly brought back to room temperature. After 6 h, the reaction was cooled to 0° C., 3.5 mL hydrogen peroxide (50 wt. % in H₂O) was added to the reaction, stirred at 0° C. for 1 h, saturated Na₂SO₃ was used to quench the reaction, the mixture was extracted with EtOAc (100 mL×2), dried the organic layer with anhydrous Na₂SO₄. The solvent was removed for column purification (0-5%, MeOH/DCM) to acquire light yellow solid (1 g). The resulting solid and 1,3-dimethylbarbitunic acid (1.44 g, 9.3 mmol) was dissolved with anhydrous DCM, degassed and charged with N₂. Pd(PPh₃)₄ (429 mg, 3.71 mmol) was added, the mixture was stirred at 35° C. for 26 h. After cooling to the room temperature, saturated NaHCO₃ was added, and extracted with DCM (150 mL×2), organic layer was washed with saturated with NaCl, dried over Na₂SO₄. After removing the solvent, the resulting solid was dissolved with 15 mL DCM, 1.37 g p-chloranil (5.57 mmol) was added portion-wise at room temperature, and the reaction stirred for another 1 h avoiding light. Load the mixture on the silica gel column using 1:1 DCM/MeOH with 1% acetic acid. After removing the solvent, the blue mixture was further purified by HPLC, water/acetonitrile with 0.1% TFA as mobile phase with ration from 95/5 to 5/95, 190 mg blue solid NR₆₄₁ (yield 12%) was acquired after removing the solvent.

¹H-NMR (600 MHz, Methanol-d₄) δ 7.57-7.45 (m, 3H), 7.45-7.39 (m, 2H), 7.16 (dt, J=7.5, 2.0 Hz, 1H), 7.04 (ddd, J=17.2, 9.3, 6.9 Hz, 2H), 6.77-6.70 (m, 2H), 4.21-4.09 (m, 2H), 2.10 (s, 1H), 2.05 (s, 2H), 1.32 (t, J=7.0 Hz, 2H), 1.28 (t, J=7.0 Hz, 1H). ¹³C-NMR (151 MHz, Methanol-d₄) δ 159.93, 159.84, 143.03, 142.96, 137.43, 137.33, 137.14, 131.62, 130.85, 129.60, 127.12, 122.06, 122.02, 118.42, 118.34, 63.76, 63.72, 19.45, 16.70, 16.65. ³¹P-NMR (201 MHz, Methanol-d₄) δ 12.09, 12.01.

### Improved Quantum Yields

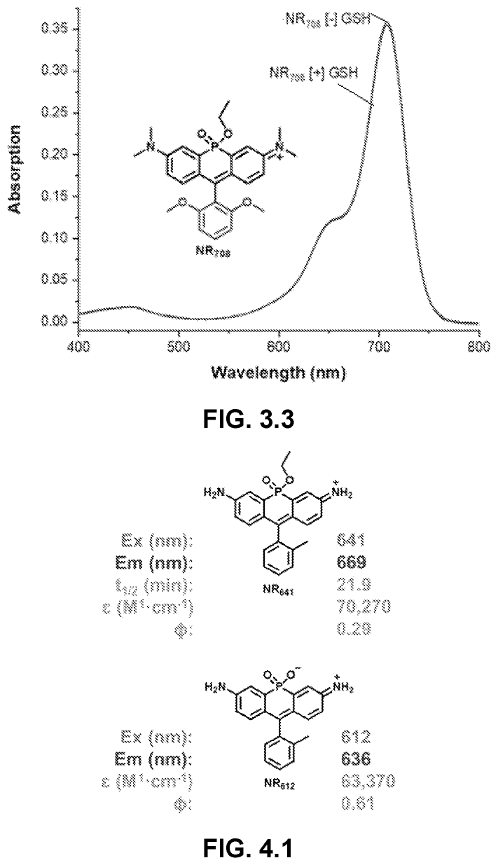

Compared to tetramethylrhodamine, rhodamine 110 derivatives bearing amine auxochromes display improved quantum yields. Accordingly, amine bearing derivatives of NR₇₀₀ and NR₆₆₆, termed NR₆₄₁ and NR₆₁₂, respectively, were synthesized (FIG. 4.1). These analogs display 2-3-fold increases in quantum yields relative to their tetramethylamino analogs, providing improved scaffolds for fluorescence imaging. Moreover, the amine substituents provide handle for further functionalization to afford new probes (e.g. for enzyme activity).

### Example 6: Phosphinate Dyes as Theranostic Agents

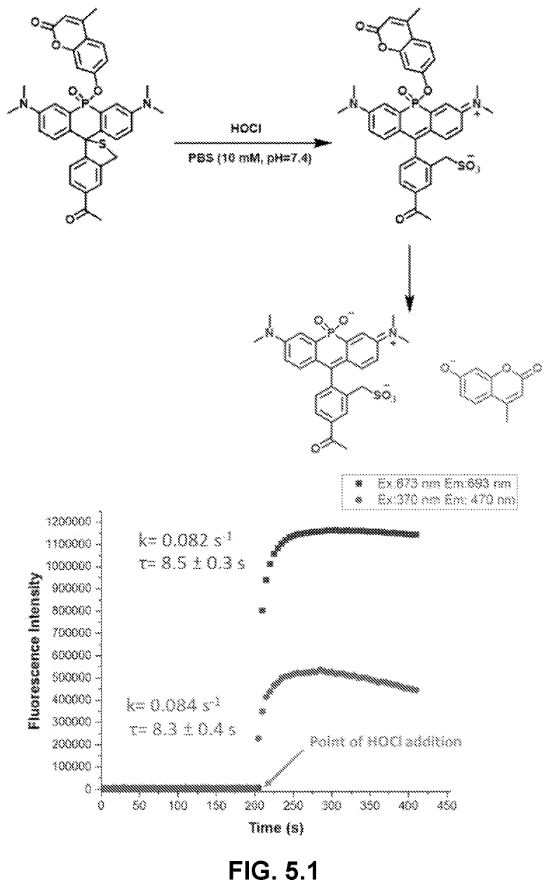

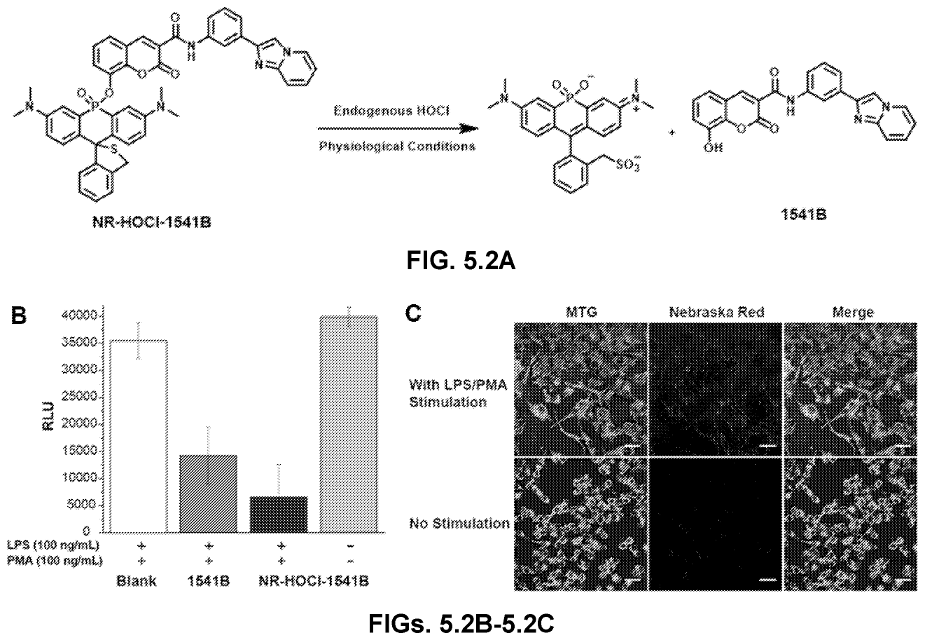

Leveraging the new chemistry afforded by the phosphinate functional group, herein is disclosed an approach in which the hydrolytic potential of phosphinate esters can be modulated by a target analyte (FIG. 1.4). It is believed that analyte-induced changes in solubility are responsible for modulating phosphinate ester hydrolysis. As a proof-of-principle, the ability to gate the delivery of coumarin, a blue fluorescent dye, from a phosphinate-containing NR dye using hypochlorous acid (HOCl) as an input, was demonstrated (FIG. 5.1). Importantly, HOCl is endogenously produced by immune cells and can be used as a selective marker for acute myeloid leukemia (AML), rheumatoid arthritis, and other diseases. Thus, molecules that could selectively target cells producing HOCl would be highly desirable. An NR-based gated delivery vehicle capable of responding to production of HOCl and producing a NIR fluorescent signal with concomitant release of a cytotoxic compound was envisioned (FIG. 5.2A). For the cytotoxic compound, 1541B, a validated activator of the executioner caspase known as caspace-3,5 a commital step in cell death, was chosen. Stimulation of macrophages to produce HOCl resulted in gating of the cytoxic effect of 1541B that was abolished in the absence of HOCl production (FIG. 5.2B, red versus pink bars). Clear evidence of gated delivery of 1541B was obtained using confocal microscopy (FIG. 5.2C). Taken together, these preliminary data clearly demonstrate the ability of the NR scaffold to respond to the altered biochemistry of a cell and simultaneously produce a fluorescent readout and deliver a small molecule agent.

### Example 7: Turn-On Photoacoustic Probes

### Synthesis and Characterization

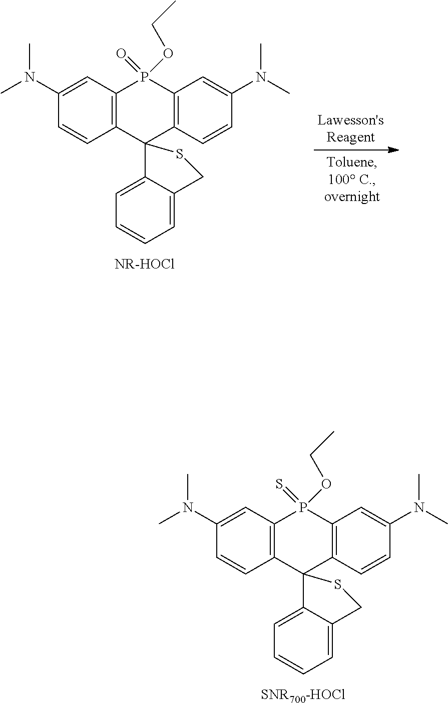

In an N₂ atmosphere, NR—HOCl (231 mg, 0.646 mmol) was dissolved in anhydrous toluene (5 mL) and evacuated/backfilled with N₂ (3×). Lawesson's reagent (402 mg, 1.292 mmol, 2 eq) was dissolved in anhydrous toluene (5 mL) and added. The reaction was allowed to proceed at 100° C. overnight. The reaction mixture was concentrated in vacuo. The residue was purified by silica gel chromatography (0-15% MeOH/DCM, linear gradient, all solutions contained 1% acetic acid) to afford SNR—HOCl (103 mg, 43%).

¹H NMR (600 MHz, Chloroform-d) δ 7.41-7.27 (m, 4H), 7.19-7.15 (m, 1H), 7.06 (dd, J=9.0, 7.5 Hz, 2H), 6.89-6.86 (m, 1H), 6.82 (dd, J=9.1, 2.9 Hz, 2H), 4.56 (s, 2H), 4.03-3.97 (m, 2H), 3.01 (s, 12H), 1.29 (t, J=7.0 Hz, 3H); ¹³C NMR (151 MHz, Chloroform-d) δ 142.68, 131.77, 129.74, 129.67, 127.74, 127.66, 127.56, 127.26, 126.51, 124.78, 124.69, 60.94, 40.36, 38.62, 37.76, 16.42, 16.22; 31P NMR (243 MHz, Chloroform-d) δ 54.38. MS: (ESI) calcd for C₂₆H₂₉N₂OPS₂ [M+H⁺] 481.2, found 481.1.

### Photoacoustic Probes

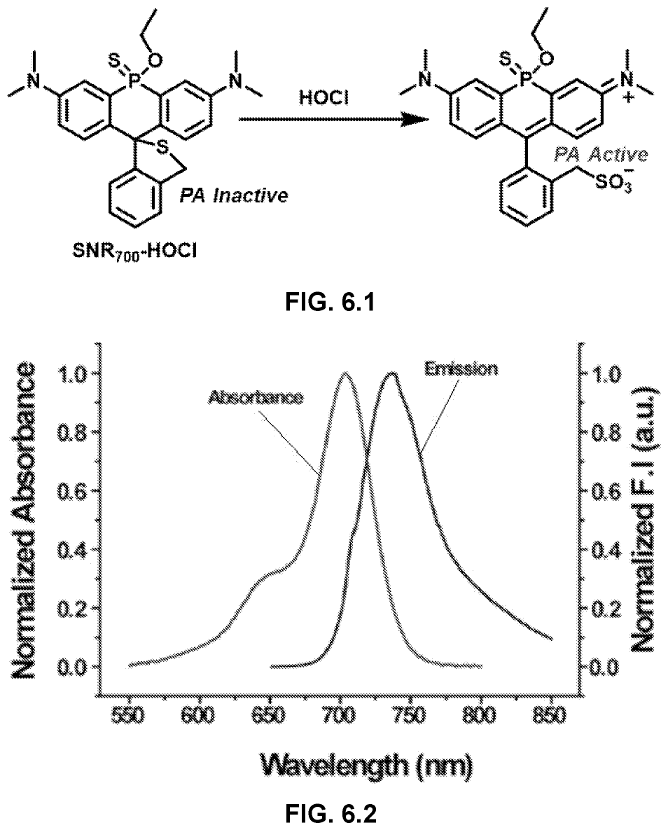

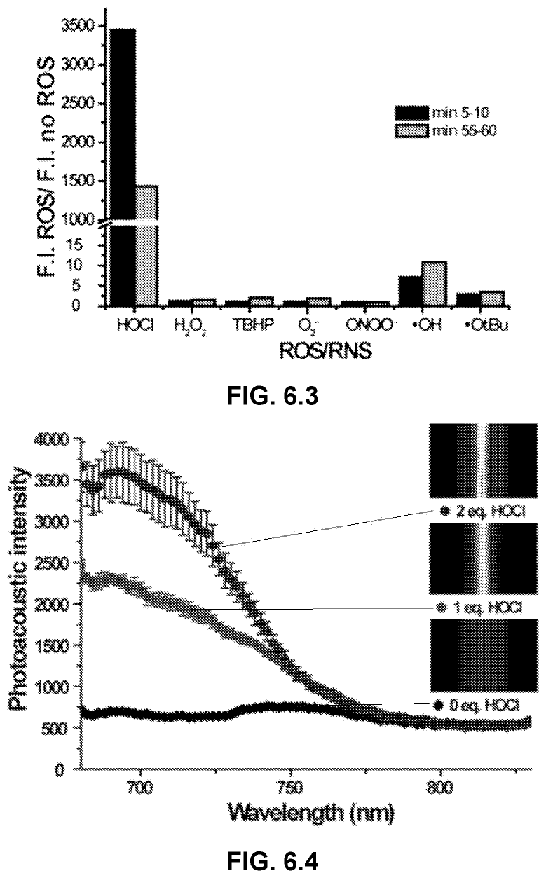

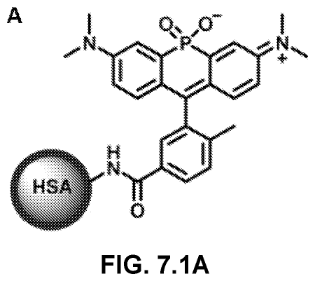

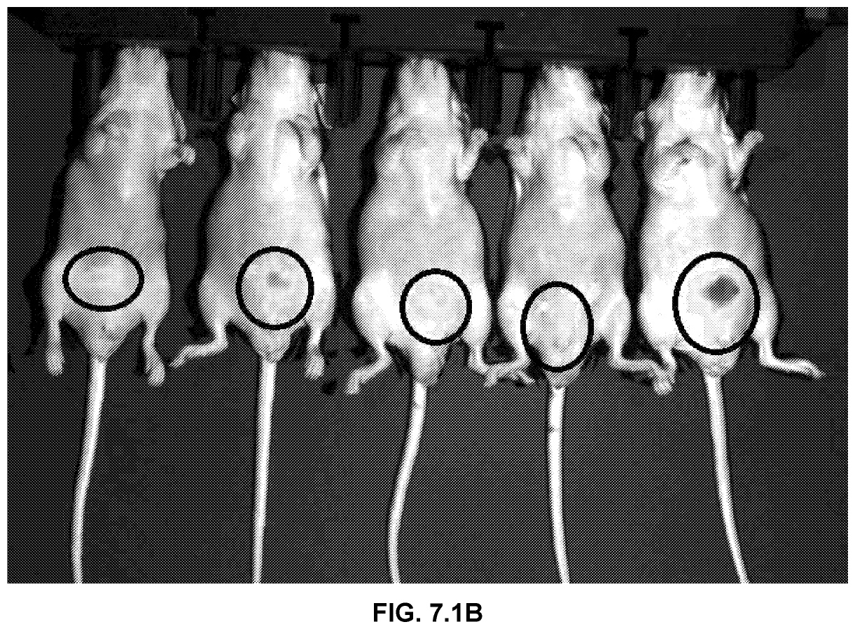

Commercial photoacoustic (PA) systems rely on excitation of an optical absorber with pulsed laser light ranging from 680-980 nm.1 Subsequent dissipation of this excitation energy leads to local and rapid heating of the sample (≤0.1° C.), resulting in thermoelastic expansion. The use of a pulsed excitation source yields fluctuating (megahertz) pressure waves that propagate through the sample as sound waves. Detection of these sound waves with a PA instrument allows for reconstruction of a 3D image of the sample. Given the wavelength retractions of PA instrumentation, whether the stabilized SNR₇₀₀ dye with maximum absorption within the range of PA instrumentation (FIG. 1.1) could be repurposed as a turn-on probe for hypochlorous acid (HOCl), a diagnostic marker of acute myeloid leukemia (AML) was investigated. Moreover, production at turn-on PA probes by leveraging the spiroring-opening detection strategy used to create turn-on absorbance and fluorescence probes from xanthene dyes was sought. Accordingly SNR₇₀₀—HOCl was designed and synthesized; this molecule contains the following features: 1) a stabilized thiophosphinate ethyl ester to maintain relatively long lived absorbance in wavelength range of commercial instrumentation and 2) spiroring recognition element for selective detection of HOCl to allow for turn-on PA signal (FIG. 6.1). Reaction with HOCl lead to a clear turn-on absorbance and fluorescence signal generation (FIG. 6.2). Turn-on signal was highly selective for HOCl compared to off-target reactive oxygen/nitrogen species (FIG. 6.3). Lastly, a clear HOCl-dependent turn-on of PA signal was observed (FIG. 6.4). Taken together, these data indicate the ability to leverage phosphinate containing dyes as turn-on PA probes, enabling noninvasive imaging of disease markers.

### Example 8: Use of NR Labeling Agents for Animal Imaging

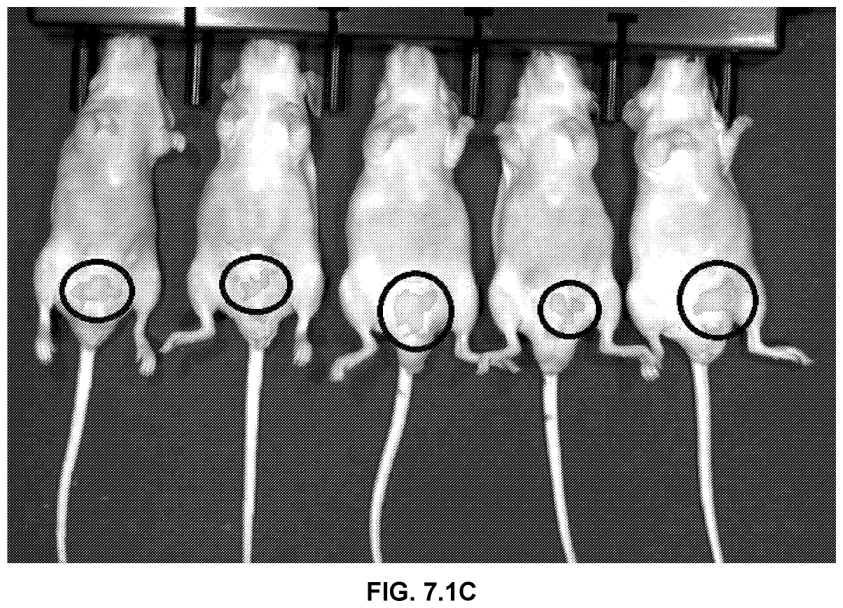

Targeted NR dyes are capable of staining tumors in preclinical cancer models. To assess the ability of NR₆₆₆ to stain tumors in a relevant animal model of disease, conjugation of NR₆₆₆ to human serum albumin (HSA) was envisioned. Importantly, HSA has previously been shown to accumulate within tumors primarily due to the enhanced permeability and retention effect (EPR). Accordingly, surface amines on HSA using 6-NHS—NR₆₆₆ to produce a NIR fluorescent probe were labeled (HSA-NR₆₆₆, FIG. 7.1A). Tail vein injection of HSA-NR₆₆₆ into mice bearing orthotopic, luciferase expressing prostate tumors and subsequent fluorescence imaging 24 hrs post-injection clearly demonstrates the ability to visualize tumors within mice using NR₆₆₆ as a reporter (FIGS. 7.1B-7.1C). NR labeling agents such as 6-NHS—NR₆₆₆ should be able to be used to produce probes for imaging as well as fluorescence-guided surgery.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Brewer, T. F.; et al. An Aza-Cope Reactivity-Based Fluorescent Probe for Imaging Formaldehyde in Living Cells. J. Am. Chem. Soc. 137, 10886-10889 (2015).
2. Chen, X., et al. Fluorescent chemosensors based on spiroring-opening of xanthenes and related derivatives. Chem. Rev. 112, 1910-1956 (2012).
3. Choi, A.; et al, Silicon Substitution in Oxazine Dyes Yields Near-Infrared Azasiline Fluorophores That Absorb and Emit beyond 700 nm. Organic letters 2018, 20 (15), 4482-4485.
4. Dohner, H., et al. Diagnosis and management of acute myeloid leukemia in adults: recommendations from an international expert panel, on behalf of the European LeukemiaNet.

### Blood 115, 453-474 (2010).

5. Fang, Y., et al. Phosphinate-containing rhodol and fluorescein scaffolds for the development of bioprobes. Chem. Commun. 55, 5962-5965 (2019).
6. Feng, H., et al. Rapid Response Fluorescence Probe Enabled In Vivo Diagnosis and Assessing Treatment Response of Hypochlorous Acid-Mediated Rheumatoid Arthritis. Adv. Sci. 5, 1800397 (2018).
7. Greish, K., Enhanced permeability and retention (EPR) effect for anticancer nanomedicine drug targeting. Methods Mol. Biol. 2010, 624, 25-37.
8. Grimm, J. B.; et al, A general method to improve fluorophores for live-cell and single-molecule microscopy. Nature methods 2015, 12 (3), 244-250.
9. Knox, H. J. et al. Acoustogenic Probes: A New Frontier in Photoacoustic Imaging. Acc Chem Res 51, 2897-2905 (2018).
10. Langer, K.; et al, Optimization of the preparation process for human serum albumin (HSA) nanoparticles. Int. J. Pharm. 2003, 257 (1-2), 169-180.
11. Li, G. P., et al. A Strategy for Highly Selective Detection and Imaging of Hypochlorite Using Selenoxide Elimination. Org Lett 15, 2002-2005 (2013).
12. Li, H., et al. Photoacoustic Probes for Ratiometric Imaging of Copper(II). J Am Chem Soc 137, 15628-15631 (2015).
13. Matsuo, T., et al. The percentage of myeloperoxidase-positive blast cells is a strong independent prognostic factor in acute myeloid leukemia, even in the patients with normal karyotype. Leukemia 17, 1538-1543 (2003).
14. Ozgen, U., et al. Further elucidation of mechanism of resistance to vincristine in myeloid cells: role of hypochlorous acid in degradation of vincristine by myeloperoxidase. Leukemia 14, 47-51 (2000).
15. Prabhakar, U.; et al, Challenges and Key Considerations of the Enhanced Permeability and Retention Effect for Nanomedicine Drug Delivery in Oncology. Cancer Res. 2013, 73 (8), 2412-2417.
16. Reinhardt, C. J. et al. Development of Photoacoustic Probes for in Vivo Molecular Imaging. Biochemistry 57, 194-199 (2018).
17. Schilling, U.; et al, Design of Compounds Having Enhanced Tumor Uptake, Using Serum-Albumin as a Carrier. Part II. In Vivo Studies. Int. J. Rad. Appl. Instrum. B 1992, 19 (6), 685-695.
18. Sinn, H.; et al, Design of compounds having an enhanced tumour uptake, using serum albumin as a carrier. Part I. Int. J. Rad. Appl. Instrum. B 1990, 17 (8), 819-827.
19. Torchilin, V., Tumor delivery of macromolecular drugs based on the EPR effect. Adv. Drug. Deliver. Rev. 2011, 63 (3), 131-135.
20. Wang, L. V. et al. A practical guide to photoacoustic tomography in the life sciences. Nat Methods 13, 627-638 (2016).
21. Wang, L. V. et al. Photoacoustic Tomography: In Vivo Imaging from Organelles to Organs. Science 335, 1458-1462 (2012).
22. Wang, S., et al. Recent Advances in Photoacoustic Imaging for Deep-Tissue Biomedical Applications. Theranostics 6, 2394-2413 (2016).
23. Weber, J., et al. Contrast agents for molecular photoacoustic imaging. Nat Methods 13, 639-650 (2016).
24. Wei, P., et al. Release of Amino- or Carboxy-Containing Compounds Triggered by HOCl: Application for Imaging and Drug Design. Angew Chem Int Edit 58, 4547-4551 (2019).
25. Wolan, D. W., et al. Small-Molecule Activators of a Proenzyme. Science 326, 853-858 (2009).
26. Zhou, X., et al. Nebraska Red: a phosphinate-based near-infrared fluorophore scaffold for chemical biology applications. Chem. Commun. 52, 12290-12293 (2016).

1. A dye having a structure of Formula I or an ionized form thereof: 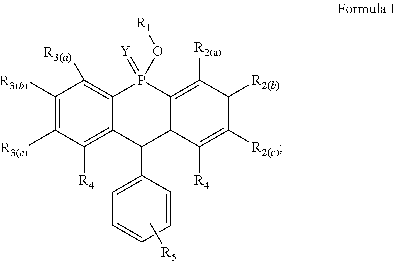
wherein R₁ is H, a C1-C6 linear or branched alkyl group, azide, alkyne, cyclooctyne, tetrazine, N-hydroxysuccinimide (NHS) ester, isocyanate, maleimide, iodoacetamide, diazonium salt, DNA-labeling agent, RNA-labeling agent, protein-labeling agent, or a 3-8-membered aryl, heteroaryl, cycloalkyl, or heterocycloalkyl group;
wherein R₂₍ₐ₎ is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{2(b)};
wherein R_{2(b)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, imine, or sulfonate, or independently forms a 5- or 6-membered aryl, cycloalkyl, or heterocycloalkyl group with R₂₍ₐ₎ or R_{2(c)}, or independently forms 5- or 6-membered aryl, cycloalkyl, or heterocycloalkyl groups with both R₂₍ₐ₎ and R_{2(c)};
wherein R_{2(c)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{2(b)};
wherein R₃₍ₐ₎ is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{3(b)};
wherein R_{3(b)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or independently forms a 5- or 6-membered aryl, cycloalkyl, or heterocycloalkyl group with R₃₍ₐ₎ or R_{3(c)}, or forms 5- or 6-membered aryl, cycloalkyl, or heterocycloalkyl groups with both R₃₍ₐ₎ and R_{3(c)};
wherein R_{3(c)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{3(b)};
wherein each R₄ is, independently, selected from hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate;
wherein each R₅ is, independently, selected from hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, C1-C4 alkoxy, amide, substituted amide, or sulfonate; and
wherein Y is O or S.

2. The dye of claim 1, wherein the structure of Formula I or ionized form thereof is selected from: 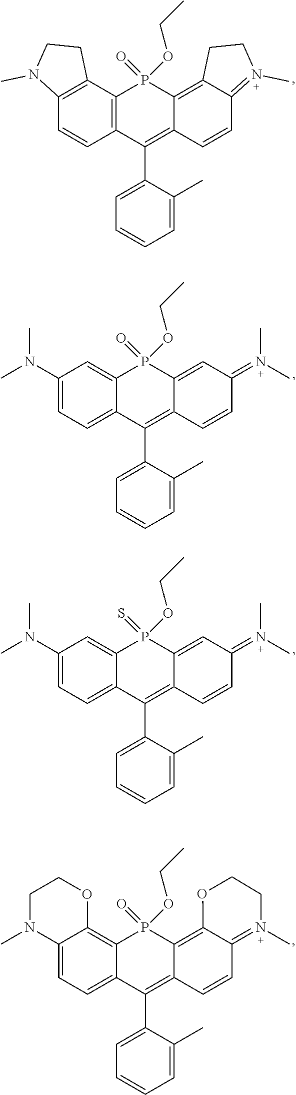 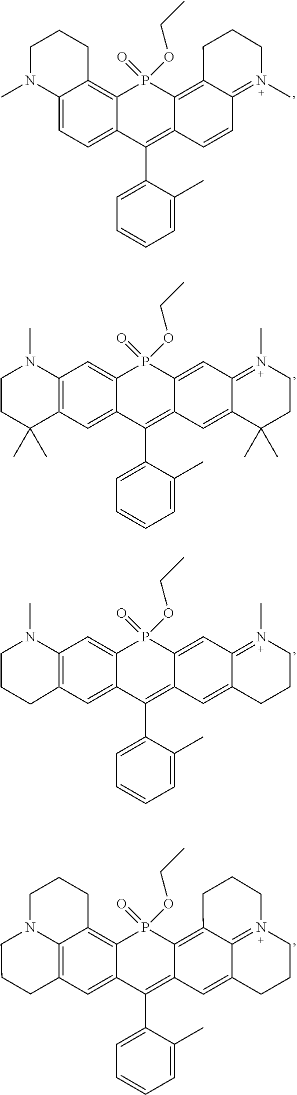 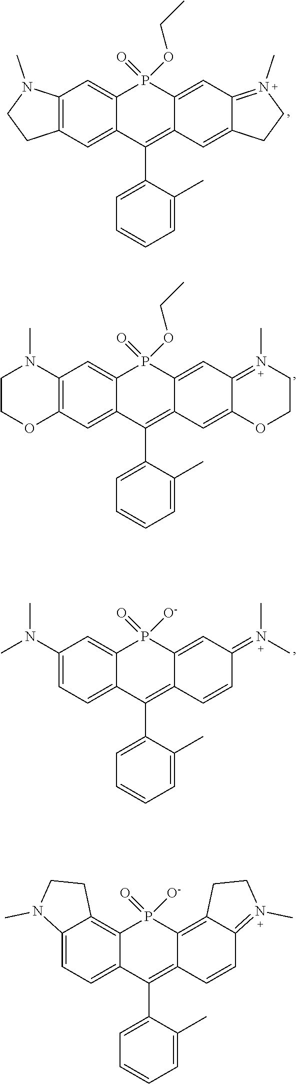 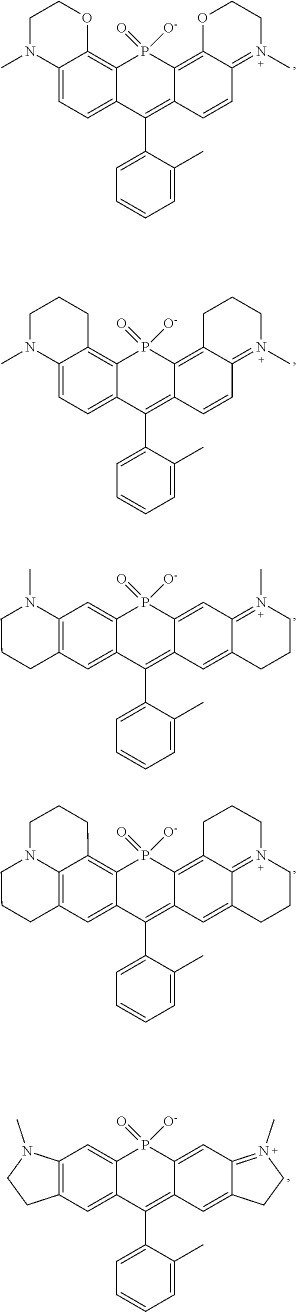 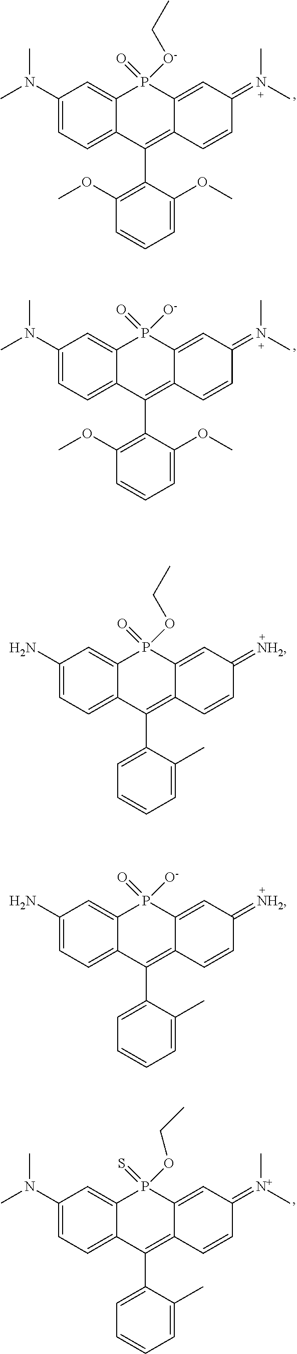
or any combination thereof.

3. The dye of claim 1, wherein the dye has an excitation wavelength of from about 610 nm to about 1500 nm and an emission wavelength maximum of from about 630 nm to about 1600 nm.

4. (canceled)

5. The dye of claim 1, wherein the dye has a half-life in water of from about 15 min to about 4 days.

6. The dye of claim 1, wherein the dye is not quenched by glutathione.

7. The dye of claim 1, wherein the dye has an extinction coefficient of from about 25,000 L/mol·cm to about 190,000 L/mol·cm.

8. A compound comprising the dye of claim 1, wherein at least one instance of R₅ comprises a substituted amide conjugated to a protein or peptide having affinity for a target molecule.

9. The compound of claim 8, wherein the compound accumulates within tumors.

10. The compound of claim 8, wherein the protein is human serum albumin, an antibody, an antibody alternative, or a growth factor.

11-12. (canceled)

13. A dye having a structure of Formula II or an ionized form thereof: 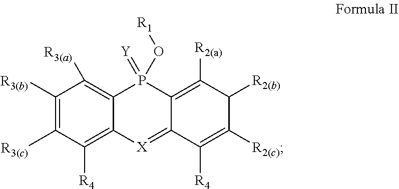
wherein R₁ is H, a C1-C6 linear or branched alkyl group, azide, alkyne, cyclooctyne, tetrazine, NHS ester, isocyanate, maleimide, iodoacetamide, diazonium salt, DNA-labeling agent, RNA-labeling agent, protein-labeling agent, or a 3-8-membered aryl, heteroaryl, cycloalkyl, or heterocycloalkyl group;
wherein R₂₍ₐ₎ is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{2(b)};
wherein R_{2(b)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, imine, azetidine, or sulfonate, or independently forms a 5- or 6-membered aryl, cycloalkyl, or heterocycloalkyl group with R₂₍ₐ₎ or R_{2(c)}, or forms 5- or 6-membered aryl, cycloalkyl, or heterocycloalkyl groups with both R₂₍ₐ₎ and R_{2(c)};
wherein R_{2(c)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{2(b)};
wherein R₃₍ₐ₎ is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, azetidine, or sulfonate, or forms a 5- or 6-membered aryl, cycloalkyl, or heterocycloalkyl group with R_{3(b)};
wherein R_{3(b)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or independently forms a 5- or 6-membered aryl, cycloalkyl, or heterocycloalkyl group with R₃₍ₐ₎ or R_{3(c)}, or forms 5- or 6-membered aryl, cycloalkyl, or heterocycloalkyl groups with both R₃₍ₐ₎ and R_{3(c)};
wherein R_{3(c)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{3(b)};
wherein each R₄ is, independently, selected from hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate;
wherein X is O, N, S, or CH; and
wherein Y is O or S.

14. The dye of claim 13, wherein the structure of Formula II or ionized form thereof is selected from: 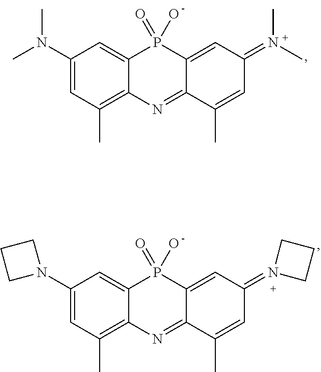
or any combination thereof.

15. The dye of claim 13, wherein the dye does not aggregate in solution.

16. The dye of claim 13, wherein the dye displays a change in emission wavelength in aqueous solution relative to emission wavelength another solvent.

17. The dye of claim 1, wherein the dye is biocompatible.

18. A compound having a structure of Formula III or an ionized form thereof: 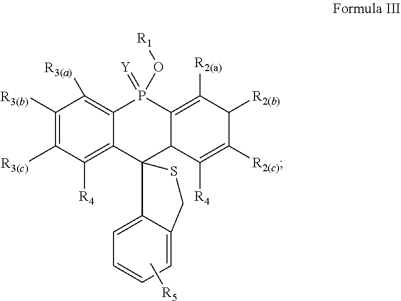
wherein R₁ is H, a C1-C6 linear or branched alkyl group, azide, alkyne, cyclooctyne, tetrazine, NHS ester, isocyanate, maleimide, iodoacetamide, diazonium salt, DNA-labeling agent, RNA-labeling agent, protein-labeling agent, or a 3-8-membered aryl, heteroaryl, cycloalkyl, or heterocycloalkyl group;
wherein R₂₍ₐ₎ is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{2(b)};
wherein R_{2(b)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, imine, or sulfonate, or independently forms a 5- or 6-membered aryl, cycloalkyl, or heterocycloalkyl group with R₂₍ₐ₎ or R_{2(c)}, or independently forms 5- or 6-membered aryl, cycloalkyl, or heterocycloalkyl groups with both R₂₍ₐ₎ and R_{2(c)};
wherein R_{2(c)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{2(b)};
wherein R₃₍ₐ₎ is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{3(b)};
wherein R_{3(b)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or independently forms a 5- or 6-membered aryl, cycloalkyl, or heterocycloalkyl group with R₃₍ₐ₎ or R_{3(c)}, or forms 5- or 6-membered aryl, cycloalkyl, or heterocycloalkyl groups with both R₃₍ₐ₎ and R_{3(c)};
wherein R_{3(c)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{3(b)};
wherein each R₄ is, independently, selected from hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate;
wherein each R₅ is, independently, selected from hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, C1-C4 alkoxy, amide, substituted amide, or sulfonate; and
wherein Y is O or S.

19. The compound of claim 18, wherein the compound comprises:

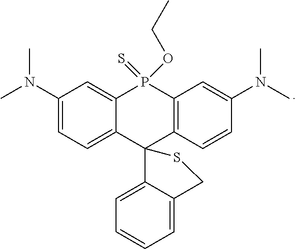

20-33. (canceled)

34. A compound having a structure of Formula IV or an ionized form thereof: 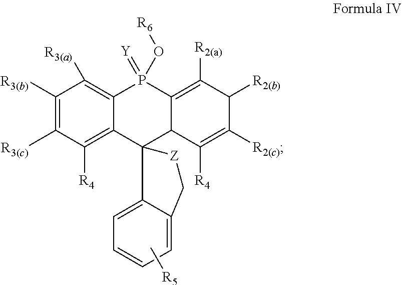
wherein R₂₍ₐ₎ is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{2(b)};
wherein R_{2(b)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, imine, or sulfonate, or independently forms a 5- or 6-membered aryl, cycloalkyl, or heterocycloalkyl group with R₂₍ₐ₎ or R_{2(c)}, or independently forms 5- or 6-membered aryl, cycloalkyl, or heterocycloalkyl groups with both R₂₍ₐ₎ and R_{2(c)};
wherein R_{2(c)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{2(b)};
wherein R₃₍ₐ₎ is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{3(b)};
wherein R_{3(b)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or independently forms a 5- or 6-membered aryl, cycloalkyl, or heterocycloalkyl group with R₃₍ₐ₎ or R_{3(c)}, or forms 5- or 6-membered aryl, cycloalkyl, or heterocycloalkyl groups with both R₃₍ₐ₎ and R_{3(c)};
wherein R_{3(c)} is hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate, or forms a 5- or 6-membered aryl, cycloalkyl, or heterocycloalkyl group with R_{3(b)};
wherein each R₄ is, independently, selected from hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, or sulfonate;
wherein each R₅ is, independently, selected from hydrogen, hydroxyl, a substituted or unsubstituted C1-C4 linear or branched alkyl group, halogen, cyano, isothiocyanate, nitro, azido, substituted or unsubstituted amino, substituted or unsubstituted alkylamino, carboxyl, C1-C4 alkoxy, amide, substituted amide, or sulfonate;
wherein Y is O or S;
wherein Z is O, S, substituted or unsubstituted amino, substituted or unsubstituted amide, substituted or unsubstituted alkyl, diazoketone, carboxyl, or a precursor thereof; and
wherein R₆ comprises a small molecule.

35. The compound of claim 34, wherein the small molecule comprises coumarin, 1541B, doxorubicin, CA4, gemcitabine, camptothecin, another chemotherapeutic agent, another cytotoxic agent, an imaging probe, or any combination thereof.

36. The compound of claim 34, wherein the compound comprises 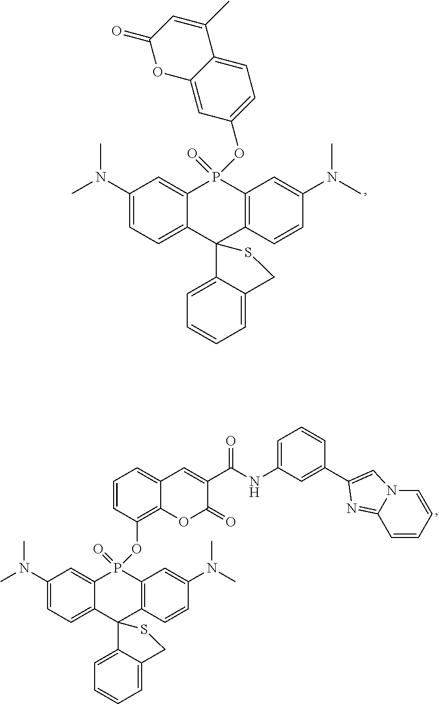
or any combination thereof.

37. The compound of any claim 34, wherein the compound is not fluorescent.

38-52. (canceled)
